(19) United States
(12) Patent Application Publication
(10) Pub. No.: US 2010/0092512 A1
(43) Pub. Date: Apr. 15, 2010

(54) Methods for preventing and ameiliorating porcine respiratory and reproductive syndrome virus-associated disease by immunizing against porcine ttv infection

(76) Inventors: John A. Ellis, Saskatoon (CA); George Steven Krakowka, Columbus, OH (US)

Correspondence Address: ROBINS & PASTERNAK, 1731 EMBARCADERO ROAD, SUITE 230, PALO ALTO, CA, 94303, US

(21) Appl. No.: 12/311,230

(22) PCT Filed: Sep. 25, 2007
(86) PCT No.: PCT/US2007/020639

§ 371 Date: Sep. 15, 2009

Related U.S. Application Data
(60) Provisional application No. 60/849,731, filed on Oct. 5, 2006.
(60) Provisional application No. 60/936,193, filed on Jun. 19, 2007.

(51) Int. Cl. A61K 39/12; C12Q 1/70; A61P 31/14

(52) U.S. Cl. 424/204.1; 435/5

(57) ABSTRACT

Compositions and methods for preventing and ameliorating Porcine respiratory and reproductive syndrome virus (PRRSV)-associated diseases in pigs by immunizing against Torque teno virus (TTV) are disclosed. Also described are methods of identifying compounds for the treatment and prevention of PRRSV-associated diseases.

### CROSS-REFERENCE TO RELATED APPLICATIONS

This application claims the benefit pursuant to 35 U.S.C. §119(e)(1) of U.S. application Ser. No. 60/849,731, filed Oct. 5, 2006, and No. 60/936,193, filed Jun. 19, 2007, which applications are incorporated herein by reference in their entireties.

### TECHNICAL FIELD

The present invention relates generally to viral pathogens. In particular, the invention pertains to Porcine respiratory and reproductive syndrome virus (PRRSV) and porcine Torque teno virus (TTV), and methods of preventing or ameliorating a PRRSV-associated disease (PRRSVD) such as interstitial pneumonia, reproductive disorders and porcine dermatitis and neuropathy syndrome (PDNS) by vaccinating pigs using TTV compositions. The invention also relates to animal models for use in studying TTV and TTV-related disorders.

### BACKGROUND

Porcine respiratory and reproductive syndrome virus (PRRSV)

Porcine respiratory and reproductive syndrome virus (PRRSV), a common swine pathogen, is an enveloped positive-stranded RNA virus (Arterivirus) that causes self-limiting respiratory disease (interstitial pneumonia) in young pigs and abortions and other reproductive disorders in pregnant swine Halbur et al., Vet Pathol. (1995) 32:200-204; Rossow, et al. Vet Pathol (1995) 32:361-373; Rossow, et al. Vet Pathol (1996) 33:551-556; Rossow, et al. Vet Pathol (1998) 35:1-20. The virus is found in most of the major pork production markets and causes millions of dollars in losses annually to the industry. As PRRSV is an RNA virus, it has a tendency to acquire either random base or recombinational mutations during its replication cycle. Thus, many different sub-strains or quasi-species of field virus, each potentially of different virulence potential, may be circulating in swine populations. Madsen et al., Arch Virol. (1998) 143:1683-1700; Meng et al., Vet Microbiol. (2000) 74:309-329; Pesch et al., Vet Microbiol. (2005) 107:31-48.

Two basic strains of PRRSV are recognized. In general the European strains of PRRSV (Leystad virus and relatives) are less virulent and not nearly as pathogenic as are the North American strains of PRRSV. Meng et al., Vet Microbiol. (2000) 74:309-329. Like all RNA viruses, there is significant genomic variation amongst strains but substantial serologic or immunologic cross-reactivity. Differences amongst the isolates are documented which, in turn appear to affect the responses of pigs to PRRSV vaccines. Madsen et al., Arch Virol. (1998) 143:1683-1700; van Woensel et al., Adv Exptl Med Biol. (1998) 440:713-718; Meng et al., Vet Microbiol. (2000) 74:309-329; Larochelle et al., Virus Res. (2003) 96:3-14; Labarque et al., Vaccine. (2004) 22:31-32; Pesch et al., Vet Microbiol. (2005) 107:31-48.

The chief manifestation of infection in young swine is an interstitial pneumonia of sufficiently distinct morphologic features that it is thought to be characteristic of infection by this agent. Halbur et al., Vet Pathol. (1995) 32:200-204; Rossow, et al., Vet Pathol (1995) 32:361-373; Rossow, et al., Vet Pathol (1996) 33:551-556; Rossow, et al., Vet Pathol (1998) 35:1-20. The viral pneumonia is characterized by septal thickening of the interstitium (not alveolar air space) with cellular infiltrates composed chiefly of monocytes and macrophages. Alveolar spaces contain fibrinonecrotic debris and macrophages and alveolar walls are lined by hyperplastic alveolar pneumocytes. There is lymphocytic perivascular cuffing in the lung as well. Alveolar and bronchial epithelia are normal. Most PRRSV pneumonias are complicated by secondary bacterial infections and the characteristic suppurative (neutrophilic) responses. In lymph nodes, prominent hyperplasia (B cell) hyperplasia is seen with necrosis of dendritic cells/macrophages in the centers of these follicles. Aside from the lung and lymph nodes, segmental lymphocytic vasculitis with occasional fibrinoid necrosis of vessel walls is reported. Similar lymphocytic infiltrates are reported in the myocardium. Renal inflammation is rarely reported to be a part of the PRRSV spectrum of lesions and hepatic lymphocytic cellular infiltrates may be seen and interpreted as PRRSV infection of Kupffer cells with a secondary inflammatory response to this phenomenon.

Porcine dermatitis and nephropathy syndrome (PDNS) has been associated with PRRSV (see, e.g., Drolet et al., Swine Health Prod. (1999) 8:283-285), as well as porcine circovirus type 2 (PCV2) (see, e.g., Allan and Ellis, J. Vet. Diag. Invest. (2000) 12:3-14), Pasteurella multocida infections (Lainson et al., J. Clin. Microbiol. (2002) 40:588-593), after exposure to bacterial endotoxins (Drolet et al., Swine Health Prod. (1999) 8:283-285), and even in swine without other identifiable infectious diseases (Segales et al. (2002) Porcine dermatitis and nephropathy syndrome in Trends in Emerging Viral Infections of Swine, eds Morilla A, Yoon K-J, Zimmerman J J (Iowa State University Press, Ames, Iowa), pp 313-318). While the incidence of PDNS is sporadic, the case fatality rate is often high. In Europe and the United Kingdom particularly, the incidence of PDNS may exceed that of PMWS (Gresham et al., Vet. Rec. (2000) 146:40-43). From studies of naturally occurring disease, young swine are more severely affected than are older swine and the clinical course in individual pigs wanes within 2-3 weeks after onset of the cutaneous lesions. PDNS-affected swine are invariably infected with PCV2 yet experimental infections of swine with PCV2 have not resulted in a single instance of PDNS in infected swine (Allan and Ellis, J. Vet. Diag. Invest. (2000) 12:3-14; Allan et al., Arch. Virol. (2000) 145:2421-2429; Krakowka et al., Vet. Pathol. (2000) 37:274-282; Krakowka et al., Vet. Pathol. (2001) 38:31-42; Krakowka et al., Virol. Immunol. (2002) 15:567-582), even when they are co-infected with PRRSV (Allan et al., Arch. Virol. (2000) 145:2421-2429; Harms et al., Vet. Pathol. (2001) 38:528-539).

Gross lesions characteristic of PDNS are distinct (see, e.g., Drolet et al., Swine Health Prod. (1999) 8:283-285). The subcutaneous hemorrhages may be confused with Rhusiopathia insidosa vasculitis or in Europe, classical or African swine fever. An accurate exclusion diagnosis for these latter two viral infectious diseases is critical. Renal lesions are diagnostic for PDNS. Grossly affected kidneys are mottled and the glomeruli of the renal cortices are accentuated as pale white-to-tan coloration or as red (hemorrhages) foci. Histologic lesions of PDNS consist of segmental necrotizing vasculitis of the dermal and sub-dermal vasculature and resultant hemorrhages and a concurrent distinct renal glomerular lesion characterized by thickening of glomerular basement membranes, protein deposits including IgG, complement and other plasma proteins, modest neutrophilic glomerular cellular infiltrates and ultimately dramatic glomerular sclerosis. Proteinuria and edema (nephrotic syndrome) are often characteristic of clinical presentation. Vascular and glomerular lesions may contain deposits of porcine IgG and complement-derived proteins; but do not contain PCV2 nucleocapsid protein or viral replicase protein nor do they hybridize with DNA probes to PCV2 viral DNAs (Segales et al. (2002) Porcine dermatitis and nephropathy syndrome in Trends in Emerging Viral Infections of Swine, eds Morilla A, Yoon K-J, Zimmerman JJ (Iowa State University Press, Ames, Iowa), pp 313-318). In spite of these negative findings, it is widely believed that PDNS is somehow an expression of PCV2 infection and PDNS is generally grouped within the PCV2-associated disease (PCVAD) or porcine circovirus disease (PCVD) complex (Allan and Ellis, J. Vet. Diag. Invest. (2000) 12:3-14; Seagales and Domingo, Vet. Quart. (2002) 24:109-124), even though the experimental evidence for cause-and-effect regarding PCV2 is lacking.

The primary in vivo cellular permissive cell(s) for PRRSV replication are monocyte macrophage lineage cells, although viral antigen has been found in myocardiocytes, myocardial endothelia and sporadically in endothelia elsewhere Halbur et al., Vet Pathol. (1995) 32:200-204; Rossow, et al., Vet Pathol (1995) 32:361-373; Rossow, et al., Vet Pathol (1996) 33:551-556; Rossow, et al., Vet Pathol (1998) 35:1-20. However, it is thought that the macrophage is the primary site of viral replication in pigs. Experimentally, the pulmonary lesions of PRRSV infection vary in severity depending upon the strain of virus involved Rossow, et al., Vet Pathol (1998) 35:1-20. The disease is commonly diagnosed by a combination of clinical signs (pneumonia and fever, with or without reproductive disease if in breeding sows), characteristic histologic changes in the lungs (see above) serology (indirect ELISA, although this test cannot distinguish between vaccinated and actively infected pigs) and reverse transcriptase (rt) PCR for PRRSV RNAs in serum samples or tissues.

It is commonly believed that PRRSV infections may become persistent in a portion of naturally infected swine in spite of apparent sero-conversion Rossow, et al., Vet Pathol (1998) 35:1-20; Murtaugh, et al., Viral Immunol (2002) 15:533-547. Virus has been recovered from the upper airways for up to four months after primary viremia and co-mingling experiments with PRRSV-convalescent and sero-negative pigs demonstrate seroconversion in the latter within two weeks suggesting that persistence is also associated with a shedding phenomenon in at least some pigs.

Swine without PRRSV infection are seronegative. In herds with stable endemic PRRSV infection, sows have low titers whereas finisher pigs show high titers indicating recent infections in the former and endemic infection in the latter. Murtaugh, et al., Viral Immunol (2002) 15:533-547. Herds that are experiencing a primary PRRSV infection seroconvert within two weeks after infection and this, along with demonstration of clinical signs of pneumonia, abortions, histologic lesions characteristic of PRRSV in the lung and demonstration of PRRSV RNA in serum by rtPCR all are used to establish a diagnosis of newly acquired PRRSV infection Christophr-Hennings et al., J Clin Micro (1995) 33:1730-1734.

Both killed and modified live vaccine (MLV) products are used to combat PRRSV infection and both are reported to be efficacious against homologous infections but of minimal efficacy against heterologous infections. Meng et al., Vet Microbiol. (2000) 74:309-329. Protection is observed as an “absence of clinical disease”, not as an “absence of virus” in vaccinates Meng et al., Vet Microbiol. (2000) 74:309-329; Murtaugh, et al., Viral Immunol (2002) 15:533-547. The problem with PRRSV and vaccinations is the issue of persistent or carrier swine and their presumed ability to serve as reservoirs for PRRSV to new generations of swine in the production facility. As well, there are frequent vaccine failures and these are invariably attributed to the evolution of unique antigenic variant strains of field PRRSV in infected herds. Mengeling et al., AJVR. (1999) 60:334-340; Meng et al., Vet Microbiol. (2000) 74:309-329; Murtaugh, et al., Viral Immunol (2002) 15:533-547. In this instance, partial protection is commonly observed. The situation of poor responses to vaccinations is further complicated by the application of MLV PRRSV vaccines to PRRSV-infected herds as the Danish experience has clearly shown that introduction of an MLV product of North American lineage spread MLV-PRRSV amongst European PRRSV strain-infected pigs, was transmissible to naïve pigs and resulted in PRRSV outbreaks in previously stable (endemically infected) Danish pig herds Madsen et al., Arch Virol. (1998) 143:1683-1700; Storgaard et al., Arch Virol. (1999) 144:2389-2401.

PRRSV infection costs the world pork industry hundreds of millions of dollars in lost revenues. Rossow, et al., Vet Pathol (1998) 35:1-20. Additionally, the annual cost of vaccinations for PRRSV is estimated at an excess of 100 million dollars, excluding labor costs and related expenses. Even more worrisome is the fact that vaccination programs are frequently inadequate. The failure of vaccination(s) to control PRRSV disease is a large concern in the industry. As explained above, these failures are attributed largely if not exclusively to the emergence of field strains of PRRSV, which do not sufficiently cross-react with vaccine virus epitope(s). Meng et al., Vet Microbiol. (2000) 74:309-329. More ominously, PRRSV vaccine viruses may actually cause disease. Madsen et al., Arch Virol. (1998) 143:1683-1700; Storgaard et al., Arch Virol. (1999) 144:2389-2401; Mengeling et al., AJVR. (1999) 60:334-340. Yet, in spite of these widespread beliefs, and in the absence of alternatives, vaccinations for PRRSV, even if of questionable efficacy, are recommended and a routine practice in the industry.

Porcine torque teno virus (TTV)

Torque teno virus (TTV), also known as transfusion-transmitted virus, belongs to the Anellovirus floating genus and has been provisionally assigned to the Circoviridae family. TTV was originally isolated from the blood of a human patient with a post-transfusion hepatitis of unknown etiology. Nishizawa et al., Biochem. Biophys. Res. Commun. (1997) 241:92-97. TTV has now been identified in a number of animals in addition to humans, including in pigs. Porcine TTV has been isolated in several countries and sequence analyses have shown that the various strains share between 71% to 100% nucleotide sequence identity. McKeown et al., Vet. Microbiol. (2004) 104:113-117.

TTV is a small, non-enveloped virus with a single-stranded circular DNA genome of negative polarity. The genome includes an untranslated region and at least three major overlapping open reading frames. Biagini, P., Vet. Micorbiol. (2004) 98:95-101. ORF1 encodes a DNA replicase, ORF2 a nucleocapsid protein and ORF3 a protein with apoptotic activity.

Porcine TTV is ubiquitous and PCR-detection of the virus in serum samples collected from various geographical regions shows prevalence in pigs ranging from 33 to 100%. McKeown et al., Vet. Microbiol. (2004) 104:113-117. To date, there is no clear relationship between porcine Try and any particular pathology. Moreover the role of TTV during co-infection with other pathogens remains unknown. Thus, TTV is considered an “orphan” virus, a virus still waiting to be clearly linked to a given disease. Beninelli et al., Clin. Microbiol. Rev. (2001) 14:98-113.

### SUMMARY OF INVENTION

From the foregoing, it is clear that PRRSV-associated interstitial pneumonia and PDNS are continuing problems in the swine industry and that attempts to quell PRRSV diseases have been largely unsuccessful. The inventors herein have surprisingly found that some PRRSV-associated respiratory conditions and PDNS may be due to primary infection or coinfection with viruses other then PRRSV, in particular TTV. Moreover, coinfection with TTV appears to worsen relatively innocuous PRRSV pulmonary infections.

Thus, the present invention relates to the use of TTV preparations in the prevention, treatment and/or diagnosis of PRRSVD, including PDNS and respiratory disease in growing and fattening pigs and reproductive disease, as well as other respiratory pathogens in swine such as swine influenza virus and mycoplasma hyopneumoniae. Attenuated, inactivated or subunit vaccines, including immunogens and mixtures of immunogens derived from porcine TTV isolates are used to provide protection against subsequent infection with TTV and are therefore useful for protection against PRRSVDs and other respiratory pathogens of swine. The present invention thus provides a commercially useful method of treating, preventing and/or diagnosing PRRSV infection in swine.

As explained above, it appears that the pathogenic effects in swine that have previously been attributed to PRRSV infections may also be due to co-infection with TTV, a newly discovered infectious agent of swine. See, U.S. Provisional Patent Application No. 60/849,731, filed Oct. 5, 2006, and PCT application entitled “Methods for Treating, Preventing and Diagnosing Porcine TTV Infection” filed on even date herewith, both of which applications are incorporated herein by reference in their entireties.

Accordingly, in one embodiment, the invention is directed to a method of preventing or ameliorating a PRRSV-associated disease (PRRSVD) in a porcine subject comprising administering to the subject a therapeutically effective amount of a composition comprising a pharmaceutically acceptable vehicle and at least one porcine TTV immunogen selected from an inactivated immunogenic porcine TTV, an attenuated immunogenic porcine TTV or one or more isolated immunogenic porcine TTV polypeptides. In certain embodiments, the composition further comprises an adjuvant. In additional embodiments, the PRRSVD is a respiratory disease, such as interstitial pneumonia, a reproductive disease, or PDNS.

In certain embodiments, the compositions used in the methods include additional immunogens from pathogens that cause disease in pigs, such as but not limited to, immunogens from porcine parvovirus, porcine circovirus, porcine reproductive and respiratory syndrome virus, swine influenza, pseudorabies virus, pestivirus which causes porcine swine fever, porcine lymphotropic herpesviruses (PLHV1 and PLHV2), Mycoplasma spp, Helicobacter spp, Campylobacter spp, Lawsonia spp, Actinobacillus pleuropneumoniae, Haemophilus parasuis, Streptococcus spp, Pasteurella spp, Salmonella spp, E. coli, Clostridium spp, Eryspelothrix rhusiopathiae.

In additional embodiments, the invention is directed to a method of determining the propensity of a porcine subject to acquire a PRRSVD, such as interstitial pneumonia, a reproductive disorder or PDNS, comprising determining whether the subject is infected with both TTV and PRRSV. As explained further herein, the inventors have found that porcine subjects infected with both TTV and PRRSV are more prone to developing interstitial pneumonia as compared to porcine subjects infected with only PRRSV.

In another embodiment, the invention is directed to a method for evaluating the ability of a vaccine to prevent a PRRSVD comprising: (a) administering to a porcine subject a candidate vaccine; (b) exposing the porcine subject from step (a) to a porcine TTV isolate and a PRRSV isolate in amounts sufficient to cause infection in an unvaccinated subject; and (c) observing the incidence of PRRSVD in the porcine subject, thereby evaluating the ability of the candidate vaccine to prevent PRRSVD.

In certain embodiments, the porcine subject is a young TTV-negative and PRRSV-negative piglet, a barrier-raised specific pathogen-free piglet, or a caesarian-delivered piglet. In additional embodiments, the PRRSVD is a respiratory and/or reproductive disease. In certain embodiments, the PRRSVD is interstitial pneumonia and/or PDNS.

In another embodiment, the invention is directed to a method of identifying a compound capable of treating a porcine PRRSVD. The method comprises (a) exposing a young TTV-negative and PRRSV-negative piglet, a barrier-raised specific pathogen-free piglet, or a caesarian-delivered piglet, to a porcine TTV isolate and a PRRSV isolate in amounts sufficient to cause infection in the piglet; (b) delivering a compound or series of compounds to the infected piglet; and (c) examining the piglet from step (b) for the presence or loss of TTV and/or the development, inhibition, or amelioration of PRRSVD symptoms relative to an untreated TTV-infected piglet.

These and other embodiments of the subject invention will readily occur to those of skill in the art in view of the disclosure herein.

### DETAILED DESCRIPTION OF THE INVENTION

The practice of the present invention will employ, unless otherwise indicated, conventional techniques of molecular biology, microbiology, bacteriology, virology, recombinant DNA technology, and immunology, which are within the skill of the art. Such techniques are explained fully in the literature. See, e.g., Sambrook, Fritsch & Maniatis, Molecular Cloning: A Laboratory Manual, current edition; Fundamental Virology, current edition, vol. I & II (B.N. Fields and D.M. Knipe, eds.); DNA Cloning, Vols. I and II (D. N. Glover ed. current edition); Oligonucleotide Synthesis (M. J. Gait ed. 1984); Nucleic Acid Hybridization (B. D. Hames & S.J. Higgins eds. 1984); Animal Cell Culture (R. K. Freshney ed. 1986); Immobilized Cells and Enzymes (IRL press, 1986); Perbal, B., A Practical Guide to Molecular Cloning (1984); the series, Methods In Enzymology (S. Colowick and N. Kaplan eds., Academic Press, Inc.); and Handbook of Experimental Immunology, Vols. I-IV (D. M. Weir and C. C. Blackwell eds., 1986, Blackwell Scientific Publications).

All publications, patents and patent applications cited herein, whether supra or infra, are hereby incorporated by reference in their entirety.

1. DEFINITIONS

In describing the present invention, the following terms will be employed, and are intended to be defined as indicated below.

It must be noted that, as used in this specification and the appended claims, the singular forms “a”, “an” and “the” include plural referents unless the content clearly dictates otherwise. Thus, for example, reference to “a TTV immunogen” includes a mixture of two or more such immunogens, and the like.

By “PRRSV infection” or “PRRSVD” is meant any disorder caused directly or indirectly by a Porcine Respiratory and Reproductive Virus, including without limitation, infection caused by any of the known PRRSV strains and isolates included in any of the PRRSV genogroups. Currently, this arterivirus is subdivided into 2 major genogroups, whose genomes diverge by approximately 40% Rossow, et al., Vet Pathol (1998) 35:1-20; Meng et al., Vet Microbiol. (2000) 74:309-329. PRRSVD include, without limitation, interstitial pneumonia; reproductive diseases such but not limited to abortion, stillbirth, mummification, return to estrus, neonatal death and failure to thrive; myocarditis; vasculitis; encephalitis and lymphadenitis. Rossow, et al., Vet Pathol (1998) 35:1-20. Porcine dermatopathy and nephropathy syndrome (PDNS) is also associated with PRRSV infection and is considered herein to be a PRRSVD Choi, Chae Vet Pathol. (2000) 38:436-41.

The term also intends subclinical disease, e.g., where PRRSV infection is present but clinical symptoms of disease have not yet manifested themselves. Subjects with subclinical disease can be asymptomatic but may nonetheless be at risk of developing any of the above disorders.

By “young piglet” is meant a piglet from birth to six weeks of age, preferably from birth to three weeks of age.

The term “polypeptide” when used with reference to a TTV or PRRSV immunogen, refers to the immunogen, whether native, recombinant or synthetic, which is derived from any TTV or PRRSV strain. The polypeptide need not include the full-length amino acid sequence of the reference molecule but can include only so much of the molecule as necessary in order for the polypeptide to retain immunogenicity and/or the ability to treat, prevent or diagnose TTV infection and PRRSV infection, as described below. Thus, only one or few epitopes of the reference molecule need be present. Furthermore, the polypeptide may comprise a fusion protein between the full-length reference molecule or a fragment of the reference molecule, and another protein that does not disrupt the reactivity of the polypeptide. It is readily apparent that the polypeptide may therefore comprise the full-length sequence, fragments, truncated and partial sequences, as well as analogs and precursor forms of the reference molecule. The term also intends deletions, additions and substitutions to the reference sequence, so long as the polypeptide retains immunogenicity.

Thus, the full-length proteins and fragments thereof, as well as proteins with modifications, such as deletions, additions and substitutions (either conservative or non-conservative in nature), to the native sequence, are intended for use herein, so long as the protein maintains the desired activity. These modifications may be deliberate, as through site-directed mutagenesis, or may be accidental, such as through mutations of hosts which produce the proteins or errors due to PCR amplification. Accordingly, active proteins substantially homologous to the parent sequence, e.g., proteins with 70 . . . 80 . . . 85 . . . 90 . . . 95 . . . 98 . . . 99% etc. identity that retain the biological activity, are contemplated for use herein.

The term “analog” refers to biologically active derivatives of the reference molecule, or fragments of such derivatives, that retain activity, as described above. In general, the term “analog” refers to compounds having a native polypeptide sequence and structure with one or more amino acid additions, substitutions and/or deletions, relative to the native molecule. Particularly preferred analogs include substitutions that are conservative in nature, i.e., those substitutions that take place within a family of amino acids that are related in their side chains. Specifically, amino acids are generally divided into four families: (1) acidic—aspartate and glutamate; (2) basic—lysine, arginine, histidine; (3) non-polar—alanine, valine, leucine, isoleucine, proline, phenylalanine, methionine, tryptophan; and (4) uncharged polar—glycine, asparagine, glutamine, cysteine, serine threonine, tyrosine. Phenylalanine, tryptophan, and tyrosine are sometimes classified as aromatic amino acids. For example, it is reasonably predictable that an isolated replacement of leucine with isoleucine or valine, an aspartate with a glutamate, a threonine with a serine, or a similar conservative replacement of an amino acid with a structurally related amino acid, will not have a major effect on the biological activity. For example, the polypeptide of interest may include up to about 5-10 conservative or non-conservative amino acid substitutions, or even up to about 15-25 or 50 conservative or non-conservative amino acid substitutions, or any number between 5-50, so long as the desired function of the molecule remains intact.

A “purified” protein or polypeptide is a protein which is recombinantly or synthetically produced, or isolated from its natural host, such that the amount of protein present in a composition is substantially higher than that present in a crude preparation. In general, a purified protein will be at least about 50% homogeneous and more preferably at least about 80% to 90% homogeneous.

By “biologically active” is meant a TTV or PRRSV protein that elicits an immunological response, as defined below.

By “subunit vaccine composition” is meant a composition containing at least one immunogen, but not all immunogens, derived from or homologous to an immunogen from TTV. Such a composition is substantially free of intact virus. Thus, a “subunit vaccine composition” is prepared from at least partially purified (preferably substantially purified) immunogens from TTV, or recombinant analogs thereof. A subunit vaccine composition can comprise the subunit antigen or antigens of interest substantially free of other antigens or polypeptides from the pathogen. Representative immunogens include those derived from any of ORFs 1, 2 or 3 of the TTV genome, such as immunogens from ORF2, the nucleocapsid protein, including the full-length protein or fragments thereof. For example, the proteins expressed from ORF2 of porcine genogroups 1 and 2 (Neil et al., J. Gen. Virol. (2005) 86:1343-1347) may both be present in the subunit composition, as well as additional immunogens from TTV or other viruses. Also encompassed is the use of consensus sequences from any of the above ORFs based on multiple genotypes of TTV, such as but not limited to TTV genotypes 1 and 2.

By “epitope” is meant a site on an antigen to which specific B cells and T cells respond. The term is also used interchangeably with “antigenic determinant” or “antigenic determinant site.” An epitope can comprise 3 or more amino acids in a spatial conformation unique to the epitope. Generally, an epitope consists of at least 5 such amino acids and, more usually, consists of at least 8-10 such amino acids. Methods of determining spatial conformation of amino acids are known in the art and include, for example, x-ray crystallography and 2-dimensional nuclear magnetic resonance. Furthermore, the identification of epitopes in a given protein is readily accomplished using techniques well known in the art, such as by the use of hydrophobicity studies and by site-directed serology. See, also, Geysen et al., Proc. Natl. Acad. Sci. USA (1984) 81:3998-4002 (general method of rapidly synthesizing peptides to determine the location of immunogenic epitopes in a given antigen); U.S. Pat. No. 4,708,871 (procedures for identifying and chemically synthesizing epitopes of antigens); and Geysen et al., Molecular Immunology (1986) 23:709-715 (technique for identifying peptides with high affinity for a given antibody). Antibodies that recognize the same epitope can be identified in a simple immunoassay showing the ability of one antibody to block the binding of another antibody to a target antigen.

An “immunological response” to a composition or vaccine is the development in the host of a cellular and/or antibody-mediated immune response to the composition or vaccine of interest. Usually, an “immunological response” includes but is not limited to one or more of the following effects: the production of antibodies, B cells, helper T cells, suppressor T cells, and/or cytotoxic T cells and/or gamma delta (γδ) T cells, directed specifically to an antigen or antigens included in the composition or vaccine of interest. Preferably, the host will display a protective immunological response to the immunogen(s) in question, e.g., the host will be protected from subsequent infection by the pathogen and such protection will be demonstrated by either a reduction or lack of symptoms normally displayed by an infected host or a quicker recovery time.

The terms “immunogenic” protein or polypeptide refer to an amino acid sequence which elicits an immunological response as described above. An “immunogenic” protein or polypeptide, as used herein, includes the full-length sequence of the particular immunogen in question, including any precursor and mature forms, analogs thereof, or immunogenic fragments thereof. By “immunogenic fragment” is meant a fragment of the immunogen in question which includes one or more epitopes and thus elicits the immunological response described above.

Immunogenic fragments, for purposes of the present invention, will usually be at lest about 2 amino acids in length, more preferably about 5 amino acids in length, and most preferably at least about 10 to 15 amino acids in length. There is no critical upper limit to the length of the fragment, which could comprise nearly the full-length of the protein sequence, or even a fusion protein comprising two or more epitopes of the immunogen in question.

“Homology” refers to the percent identity between two polynucleotide or two polypeptide moieties. Two DNA, or two polypeptide sequences are “substantially homologous” to each other when the sequences exhibit at least about 50%, preferably at least about 75%, more preferably at least about 80%-85%, preferably at least about 90%, and most preferably at least about 95%-98% sequence identity over a defined length of the molecules. As used herein, substantially homologous also refers to sequences showing complete identity to the specified DNA or polypeptide sequence.

In general, “identity” refers to an exact nucleotide-to-nucleotide or amino acid-to-amino acid correspondence of two polynucleotides or polypeptide sequences, respectively. Percent identity can be determined by a direct comparison of the sequence information between two molecules by aligning the sequences, counting the exact number of matches between the two aligned sequences, dividing by the length of the shorter sequence, and multiplying the result by 100. Readily available computer programs can be used to aid in the analysis, such as ALIGN, Dayhoff, M. O. in Atlas of Protein Sequence and Structure M.O. Dayhoff ed., 5 Suppl. 3:353-358, National Biomedical Research Foundation, Washington, D.C., which adapts the local homology algorithm of Smith and Waterman Advances in Appl. Math. 2:482-489, 1981 for peptide analysis. Programs for determining nucleotide sequence identity are available in the Wisconsin Sequence Analysis Package, Version 8 (available from Genetics Computer Group, Madison, Wis.) for example, the BESTFIT, FASTA and GAP programs, which also rely on the Smith and Waterman algorithm. These programs are readily utilized with the default parameters recommended by the manufacturer and described in the Wisconsin Sequence Analysis Package referred to above. For example, percent identity of a particular nucleotide sequence to a reference sequence can be determined using the homology algorithm of Smith and Waterman with a default scoring table and a gap penalty of six nucleotide positions. Another method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs are well known in the art.

Alternatively, homology can be determined by hybridization of polynucleotides under conditions which form stable duplexes between homologous regions, followed by digestion with single-stranded-specific nuclease(s), and size determination of the digested fragments. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; DNA Cloning, supra; Nucleic Acid Hybridization, supra.

A “coding sequence” or a sequence which “encodes” a selected polypeptide, is a nucleic acid molecule which is transcribed (in the case of DNA) and translated (in the case of mRNA) into a polypeptide in vitro or in vivo when placed under the control of appropriate regulatory sequences. The boundaries of the coding sequence are determined by a start codon at the 5′ (amino) terminus and a translation stop codon at the 3′ (carboxy) terminus. A transcription termination sequence may be located 3′ to the coding sequence.

By “vector” is meant any genetic element, such as a plasmid, phage, transposon, cosmid, chromosome, virus, virion, etc., which is capable of replication when associated with the proper control elements and which can transfer gene sequences to cells. Thus, the term includes cloning and expression vehicles, as well as viral vectors.

By “recombinant vector” is meant a vector that includes a heterologous nucleic acid sequence which is capable of expression in vitro or in vivo.

The term “transfection” is used to refer to the uptake of foreign DNA by a cell, and a cell has been “transfected” when exogenous DNA has been introduced inside the cell membrane. A number of transfection techniques are generally known in the art. See, e.g., Graham et al. (1973) Virology, 52 :456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous DNA moieties into suitable host cells.

The term “heterologous” as it relates to nucleic acid sequences such as coding sequences and control sequences, denotes sequences that are not normally joined together, and/or are not normally associated with a particular cell. Thus, a “heterologous” region of a nucleic acid construct or a vector is a segment of nucleic acid within or attached to another nucleic acid molecule that is not found in association with the other molecule in nature. For example, a heterologous region of a nucleic acid construct could include a coding sequence flanked by sequences not found in association with the coding sequence in nature. Another example of a heterologous coding sequence is a construct where the coding sequence itself is not found in nature (e.g., synthetic sequences having codons different from the native gene). Similarly, a cell transformed with a construct which is not normally present in the cell would be considered heterologous for purposes of this invention. Allelic variation or naturally occurring mutational events do not give rise to heterologous DNA, as used herein.

A “nucleic acid” sequence refers to a DNA or RNA sequence. The term captures sequences that include any of the known base analogues of DNA and RNA such as, but not limited to 4-acetylcytosine, 8-hydroxy-N6-methyladenosine, aziridinylcytosine, pseudoisocytosine, 5-(carboxyhydroxyl-methyl) uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudo-uracil, 1-methylguanine, 1-methylinosine, 2,2-dimethyl-guanine, 2-methyladenine, 2-methylguanine, 3-methyl-cytosine, 5-methylcytosine, N6-methyladenine, 7-methylguanine, 5-methylaminomethyluracil, 5-methoxy-amino-methyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, -uracil-5-oxyacetic acid methylester, uracil-5-oxyacetic acid, pseudouracil, queosine, 2-thiocytosine, and 2,6-diaminopurine.

The term DNA “control sequences” refers collectively to promoter sequences, polyadenylation signals, transcription termination sequences, upstream regulatory domains, origins of replication, internal ribosome entry sites (“IRES”), enhancers, and the like, which collectively provide for the replication, transcription and translation of a coding sequence in a recipient cell. Not all of these control sequences need always be present so long as the selected coding sequence is capable of being replicated, transcribed and translated in an appropriate host cell.

The term “promoter” is used herein in its ordinary sense to refer to a nucleotide region comprising a DNA regulatory sequence, wherein the regulatory sequence is derived from a gene which is capable of binding RNA polymerase and initiating transcription of a downstream (3′-direction) coding sequence. Transcription promoters can include “inducible promoters” (where expression of a polynucleotide sequence operably linked to the promoter is induced by an analyte, cofactor, regulatory protein, etc.), “repressible promoters” (where expression of a polynucleotide sequence operably linked to the promoter is induced by an analyte, cofactor, regulatory protein, etc.), and “constitutive promoters”.

“Operably linked” refers to an arrangement of elements wherein the components so described are configured so as to perform their usual function. Thus, control sequences operably linked to a coding sequence are capable of effecting the expression of the coding sequence. The control sequences need not be contiguous with the coding sequence, so long as they function to direct the expression thereof. Thus, for example, intervening untranslated yet transcribed sequences can be present between a promoter sequence and the coding sequence and the promoter sequence can still be considered “operably linked” to the coding sequence.

For the purpose of describing the relative position of nucleotide sequences in a particular nucleic acid molecule throughout the instant application, such as when a particular nucleotide sequence is described as being situated “upstream,” “downstream,” “3 prime (3′)” or “5 prime (5′)” relative to another sequence, it is to be understood-that it is the position of the sequences in the “sense” or “coding” strand of a DNA molecule that is being referred to as is conventional in the art.

The terms “effective amount” or “therapeutically effective amount” of a composition or agent, as provided herein, refer to a nontoxic but sufficient amount of the composition or agent to provide the desired “therapeutic effect,” such as to elicit an immune response as described above, preferably preventing, reducing or reversing symptoms associated with the TTV and PRRSV infection. This effect can be to alter a component of a disease (or disorder) toward a desired outcome or endpoint, such that a subject's disease or disorder shows improvement, often reflected by the amelioration of a sign or symptom relating to the disease or disorder. For example, a representative therapeutic effect can render the subject negative for TTV infection when samples from pigs are cultured for TTV. Similarly, biopsies indicating lowered IgG, IgM and IgA antibody production directed against TTV can be an indication of a therapeutic effect. Similarly, decreased serum antibodies against TTV are indicative of a therapeutic effect. Reduced symptoms of PRRSVD-related disease are also indicative of a therapeutic effect. The exact amount required will vary from subject to subject, depending on the species, age, and general condition of the subject, the severity of the condition being treated, and the particular components of the composition administered, mode of administration, and the like. An appropriate “effective” amount in any individual case may be determined by one of ordinary skill in the art using routine experimentation.

“Treatment” or “treating” PRRSV infection includes: (1) preventing the PRRSVD, or (2) causing PRRSVD to develop or to occur at lower rates, (3) reducing the amount of TTV present in a subject, and/or reducing the symptoms associated with PRRSVD.

As used herein, a “biological sample” refers to a sample of tissue or fluid isolated from an individual, including but not limited to, for example, blood, plasma, serum, fecal matter, urine, bone marrow, bile, spinal fluid, lymph fluid, samples of the skin, external secretions of the skin, respiratory, intestinal, and genitourinary tracts, tears, saliva, milk, blood cells, organs, biopsies and also samples of in vitro cell culture constituents including but not limited to conditioned media resulting from the growth of cells and tissues in culture medium, e.g., recombinant cells, and cell components.

As used herein, the terms “label” and “detectable label” refer to a molecule capable of detection, including, but not limited to, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like. The term “fluorescer” refers to a substance or a portion thereof which is capable of exhibiting fluorescence in the detectable range. Particular examples of labels which may be used under the invention include fluorescein, rhodamine, dansyl, umbelliferone, Texas red, luminol, acradimum esters, NADPH and α-β-galactosidase.

2. MODES OF CARRYING OUT THE INVENTION

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

Central to the present invention is the use of TTV and immunogens derived therefrom, to develop immunogenic compositions for use as vaccines and diagnostics to prevent, treat and diagnose TTV and PRRSV infection. As explained above, the inventors herein have discovered that TTV is associated with PRRSVD-associated diseases. Thus, the invention provides compositions and methods for preventing, treating and diagnosing a PRRSVD.

In particular, the inventors herein have devised a method to exclude PRRSV infection from a TTV inoculum by a combination of chloroform extractions designed to inactivate and destroy PRRSV using barrier-raised swine. Feinstein et al., Infect Immun. (1983) 41:816-821. Further, the inventors have found that TTV infection in these swine produces interstitial pneumonic lesions grossly and histologically indistinguishable from those attributed to PRRSV infection. Moreover, TTV, when combined with PRRSV infection potentiates the viral pneumonia seen and increases the mortality rate from 0% to 10-15% in dually infected swine. Thus, a method of functional isolation of TTV has been devised by combining biochemical and physical exclusions of PRRSV. Using this TTV inoculum, the inventors have consistently reproduced an interstitial pneumonia in swine that remain PRRSV-negative as determined by both serology (indirect ELISA-negative) and virology (rtPCR-negative) diagnostics. These swine, however, develop histologic lesions in the lung indistinguishable from those ascribed to PRRSV. Thus, it appears that TTV infection is another prominent cause of viral interstitial pneumonia and that a vaccine product(s) developed for this agent will reduce or eliminate interstitial viral pneumonia in swine, independent of their PRRSV infection and vaccination status. Moreover, swine immunized and clinically protected from TTV should have minimal clinical disease and lesions upon subsequent infection with TTV and/or PRRSV. In other words swine immunologically protected from TTV should display minimal PRRSVD.

Moreover, plasma samples were collected from clinically asymptomatic pigs from a high health pig herd during the early phases of a disease outbreak for experimental transmission of disease into gnotobiotic swine. By the second in vivo pass, all inoculated gnotobiotes developed bilaterally symmetrical subcutaneous hemorrhages in the abdomen and over the hams and expressed severe systemic coagulation defects with associated generalized edema and icterus. At termination, in addition to hemorrhages in the dermis, anemia, edema, icterus and variable mottled and discolored livers and the gross and histologic lesions in kidneys typical of PDNS were observed. A similar disease syndrome was experimentally reproduced in additional groups of gnotobiotic swine co-infected with porcine genogroup 1 TTV and PRRSV. Gross and histologic lesions compatible with PDNS were seen only in piglets infected with both TTV and PRRSV and not PRRSV, implicating dual infections with TTV and PRRSV in the genesis of these lesions.

Animal models can be used to study the pathogenesis, treatment and prevention of PRRSV infection in pigs. For example, young TTV-negative and PRRSV-negative piglets, barrier-raised specific pathogen-free piglets, or caesarian-delivered piglets can be used to study the ability of various TTV vaccines to prevent PRRSV infection. Additionally, young TTV-negative and PRRSV-negative piglets, barrier-raised specific pathogen-free piglets, or caesarian-delivered piglets infected with TTV and PRRSV can be used to screen various compounds for their ability to treat PRRSV infection, such as interstitial pneumonia.

In order to further an understanding of the invention, a more detailed discussion is provided below regarding animal models, TTV immunogens, as well as various uses thereof.

Animal Models

Swine are monogastric omnivores with gastric anatomy and physiology that closely replicates humans. Young TTV-negative and PRRSV piglets, barrier-raised specific pathogen-free piglets, or caesarian-delivered piglets may be suited for studying PRRSV infection and therefore for identifying vaccine candidates, such as vaccines including one or more immunogens derived from TTV, useful for preventing PRRSV infection in pigs. Barrier-born pigs are free from the specific pathogens affecting individual herds. Caesarian and barrier derived animals have been shown to have a markedly reduced prevalence of porcine diseases. See, e.g., Tucker et al., Xenotransplantation (2003) 10: 343-348.

Thus, a preferred use for the animal models of the invention is the development of vaccines for use in the prevention and/or treatment of PRRSVD in pigs, such as interstitial pneumonia.

In this context, young TTV-negative and PRRSV-negative piglets, barrier-raised specific pathogen-free piglets, or caesarian-delivered piglets are administered the vaccine candidate at least once, and preferably boosted with at least one additional immunization. For example, piglets can be administered a vaccine composition to be tested at 1-5 days of age, followed by a subsequent boost 5-10 days later, and optionally a third immunization 5-10 days following the second administration. Piglets can be vaccinated as many times as necessary. The vaccinated piglets are then exposed to TTV and PRRSV approximately 3-20 days later, such as 4-10 days following the last immunization. Pigs are generally exposed to TTV first, but can be exposed in any order. Typically, vaccinated piglets are parenterally or orally administered from 10² to 10⁸ pfu, more particularly from 10⁵ to 10⁷ pfu of virus, and indicia of infection are monitored.

Such indicia include TTV viral titer, as well as symptoms of PRRSVD, such as symptoms of respiratory and reproductive problems, the presence of PRRSV antigen, and histopathology and PRRSV detection by in situ hybridization. See, e.g., Halbur et al., Vet Pathol. (1995) 32:200-204; Rossow, et al. Vet Pathol (1995) 32:361-373; Rossow, et al. Vet Pathol (1996) 33:551-556; Rossow, et al. Vet Pathol (1998) 35:1-20. Similarly, signs of PDNS can be monitored. The most obvious signs are red-purple blotches on the skin of pigs that are often slightly raised. Such blotches tend to be most obvious on the hind legs, loin, scrotum and ears, but can extend over the abdomen, flanks and fore legs eventually covering the whole body. The lesions become crusty and brown after a few days. Pigs are lethargic and elevated temperatures may also be present. In acute cases, pigs have swollen legs leading to lameness. Respiratory distress and/or scouring may also be observed. Renal lesions are also diagnostic for PDNS. Grossly affected kidneys are mottled and the glomeruli of the renal cortices are accentuated as pale white-to-tan coloration or as red (hemorrhages) foci. Histologic lesions of PDNS consist of segmental necrotizing vasculitis of the dermal and sub-dermal vasculature and resultant hemorrhages and a concurrent distinct renal glomerular lesion characterized by thickening of glomerular basement membranes, protein deposits including IgG, complement and other plasma proteins, modest neutrophilic glomerular cellular infiltrates and ultimately dramatic glomerular sclerosis. Proteinuria and edema (nephrotic syndrome) are often characteristic of clinical presentation. Vascular and glomerular lesions may contain deposits of porcine IgG and complement-derived proteins.

Alternatively, young TTV-negative and PRRSV-negative piglets, barrier-raised specific pathogen-free piglets, or caesarian-delivered piglets can first be infected with TTV in order to establish TTV infection and subsequently with PRRSV. For example, piglets can be parenterally inoculated at 1-5 days of age with TTV, in an amount sufficient to cause infection. Piglets are subsequently inoculated 1-10 days after the initial TTV exposure, more preferably 3-7 days after TTV exposure and most preferably 4-5 days after TTV exposure. Conversely, piglets can first be infected with PRRSV and subsequently infected with TTV at time periods as described above. Alternatively, piglets can be concurrently infected with TTV and PRRSV, either in the same or in different compositions. Typically, a sample will be sufficient to infect a piglet if virus can be detected using a PCR technique. Once the piglets are infected, the presence of viral infection can similarly be confirmed by examining biological samples, such as serum samples, for virus, e.g., using any number of techniques, including by ELISA, PCR, RT-PCR, Immune Fluorescence or IPMA tests, and virus isolation, all well known in the art. See, e.g., Done et al., Br. vet. J. (1996):152-153; Oleksiewicz et al., Vet. Microbiol. 64:7-22. Additionally, pigs can be monitored for signs of infection as described above.

Once the viral infection has been established, a compound or a series of compounds can be delivered to the infected piglet at various times and in various dosages, depending on the particular goals of the screen. In a variation of this procedure, it may be desirable to administer TTV and/or PRRSV with a compound to determine whether, relative to control animals, the compound can effectively prevent in vivo the initial viral infection and/or the subsequent establishment of infection or pathogenesis.

Thus, the infected piglets can be used to screen for compounds and conditions which prevent PRRSVD, such as compounds and conditions that block entry of TTV into host cells and/or that ameliorate the TTV-associated pathogenesis seen with PRRSV-associated diseases. The efficacy of the compound or compounds can be assessed by examining at selected times biological samples from the infected animals for the presence or loss of TTV and/or the development, inhibition, or amelioration of PRRSV-associated lesions relative to appropriate control animals, for example, untreated TTV-infected animals. The animal models described herein therefore provide the ability to readily assess the efficacy of various drugs or compounds based on different modes of administration and compound formation.

In addition to using the TTV-infected and PRRSV-infected animals to screen for therapeutic compounds, these animals can also be used to screen for conditions or stimuli which effect a block in or ameliorate PRRSVD. Such stimuli or conditions include environmental or dietary changes, or combinations of various stimuli or conditions which result in stress on the animal. Thus, for example, TTV-infected and PRRSV-infected animals can be exposed to a selected stimulus or condition, or a combination of stimuli or conditions, to be tested. Biological samples of exposed animals are then examined periodically for a change in the number of TTV and PRRSV and/or the associated disease state relative to non-exposed control animals.

TTV Compositions

The animal models described herein can be used to identify TTV immunogenic compositions, useful for diagnosing, treating and/or preventing PRRSVD in swine. As explained above, TTV compositions for use as vaccines and diagnostics can include attenuated or inactivated virus. Alternatively, subunit compositions, including isolated TTV immunogens, such as immunogens derived from any of the ORFs, particularly the nucleocapsid protein, encoded by ORF2 of the virus, can also be provided. Immunogens from ORF1 and ORF3 may also find use herein. Proteins including consensus sequences derived from multiple genogroups, such as TTV porcine genogroups 1 and 2, can also be used.

TTV compositions may be derived from any TTV strain and isolate in any of the TTV genogroups. A number of TTVs are known and described in, e.g., Biagini et al., “Anellovirus,” p. 335-341 in Fauquet et al. eds. Virus taxonomy, 8th report of the International Committee for the Taxonomy of Viruses. Elsevier/Acadmeic Press, New York (2004); Devalle and Niel, J. Med. Virol. (2004) 72:166-173; Hino, S., Rev. Med. Virol. (2002) 12:151-158; Nishizawa et al., Biochem. Biophys. Res. Commun. (1997) 241:92-97; Okamoto and Mayumi, J. Gastroenterol. (2001) 36:519-529; and Peng et al., Arch. Virol. (2002) 147:21-41.

Particular porcine isolates include but are not limited to Sd-TTV31, Sd-TTV1p, Sd-TTV2p, TTV isolates 3h and 2h (see, e.g., Niel et al., J. Gen. Virol. (2005) 86:1343-1347; Okamoto et al., J. Gen. Virol. (2002) 83:1291-1297). The genomic sequences of these isolates, including the sequences of ORF1, ORF2 and ORF3, encoding the DNA replicase, nucleocapsid protein and an apoptotic sequence, respectively, are described in, for example, Niel et al., J. Gen. Virol. (2005) 86:1343-1347; Okamoto et al., J. Gen. Virol. (2002) 83:1291-1297, as well as in NCBI Accession nos. AB076001, AY823991, AY823990, AY823989 and AY823988. Accession nos. DQ229865 and DQ229860 provide the sequences of porcine genogroup 1 TTV and porcine genogroup 2 TTV, respectively.

TTV immunogens, including whole TTV virus, can be produced using a variety of techniques. For example, the immunogens can be obtained directly from TTV that has been isolated from TTV-infected subjects, such as swine, using techniques well known in the art. Generally, TTV DNA is obtained using polymerase chain reaction (PCR) techniques, including TaqMan™ methods, using primers derived from the TTV genomic sequence, as described in Desai et al., J. Med. Virol. (2005) 77:136-143; Haramoto et al., Water Res. (2005) 39:2008-2013; Kekarainen et al., J. Gen. Virol. (2006) 87:833-837; and Martelli et al., J. Vet. Med. (2006) 53:234-238.

TTV so obtained can be replicated in various cell lines, such as hepatocyte and leukocyte cell lines, including the Chang Liver cell line, phytohemagllutinin (PHA)-stimulated peripheral blood mononuclear cell (PBMC) cultures and B lymphoblast cell lines, such as Raji, L23, L35, LCL 13271 cell lines. See, e.g., Desai et al., J. Med. Virol. (2005) 77:136-143; Bonenfant et al., Xenotransplantation (2003) 10:107-119; and Sinkora et al., Vet. Immunol. Immunopath. (2001) 80:79-91). Culture conditions for the above cell types are described in a variety of publications. The cell culture conditions to be used for the desired application (temperature, cell density, pH value, etc.) are variable over a very wide range depending on the cell line employed and can readily be adapted to the requirements of the TTV virus in question. Methods for propagating TTV in cultured cells include the steps of inoculating the cultured cells with TTV, cultivating the infected cells for a desired time period for virus propagation, such as for example as determined by virus titer or virus antigen expression (e.g., between 24 and 168 hours after inoculation) and collecting the propagated virus. The cultured cells are inoculated with the desired virus (measured by PFU or TCID50) to cell ratio of 1:500 to 1:1, preferably 1:100 to 1:5, more preferably 1:50 to 1:10. The TTV is added to a suspension of the cells or is applied to a monolayer of the cells, and the virus is absorbed on the cells for at least 60 minutes but usually less than 300 minutes, preferably between 90 and 240 minutes at 25° C. to 40° C., preferably 28° C. to 37° C. The infected cell culture (e.g., monolayers) may be removed either by freeze-thawing or by enzymatic action to increase the viral content of the harvested culture supernatants. The harvested fluids are then either inactivated or stored frozen.

Methods of inactivating or killing viruses are known in the art. Such methods destroy the ability of the viruses to infect mammalian cells. Inactivation can be achieved using either chemical or physical means. Chemical means for inactivating TTV include treatment of the virus with an effective amount of one or more of the following agents: detergents, formaldehyde, formalin, β-propiolactone, or UV light. Other methods of viral inactivation are known in the art, such as for example binary ethylamine, acetyl ethyleneimine, or gamma irradiation.

For example, β-propiolactone may be used at concentrations such as 0.01 to 0.5%, preferably at 0.5% to 0.2%, and still more preferably at 0.025 to 0.1%. The inactivating agent is added to virus-containing cultures (virus material) prior to or after harvesting. The cultures can be used directly or cells disrupted to release cell-associated virus prior to harvesting. Further, the inactivating agent may be added after cultures have been stored frozen and thawed, or after one or more steps of purification to remove cell contaminants. β-propiolactone is added to the virus material, with the adverse shift in pH to acidity being controlled with sodium hydroxide (e.g., 1 N NaOH) or sodium bicarbonate solution. The combined inactivating agent-virus materials are incubated at temperatures from 4° C. to 37° C., for incubation times of preferably 24 to 72 hours.

Alternatively, binary ethyleneimine (BEI) can be used to inactivate virus. One representative method of inactivating TTV is as follows. BEI is made by mixing equal volumes of a 0.2 molar bromoethylamine hydrobromide solution with a 0.4 molar sodium hydroxide solution. The mixture is incubated at about 37° C. for 60 minutes. The resulting cyclized inactivant, BEI, is added to the virus materials at 0.5 to 4 percent, and preferably at 1 to s3 percent, volume to volume. The inactivating virus materials are held from about 4° C. to 37° C. for 24 to 72 hours with periodic agitation. At the end of this incubation, 20 ml of a sterile 1 molar sodium thiosulfate solution is added to insure neutralization of the BEI. Diluted and undiluted samples of the inactivated virus materials are added to susceptible cell (tissue) culture to detect any non-inactivated virus. The cultured cells are passaged multiple times and examined for the presence of TTV based on any of a variety of methods, such as, for example, cytopathic effect (CPE) and antigen detection. Such tests allow determination of complete virus inactivation.

Methods of purification of inactivated virus are known in the art and may include one or more of gradient centrifugation, ultracentrifugation, continuous-flow ultracentrifugation and chromatography, such as ion exchange chromatography, size exclusion chromatography, and liquid affinity chromatography. Other examples of purification methods suitable for use in the invention include polyethylene glycol or ammonium sulfate precipitation, as well as ultrafiltration and microfiltration.

The purified viral preparation of the invention is substantially free of contaminating proteins derived from the cells or cell culture and preferably comprises less than about 50 pg cellular nucleic acid/μg virus antigen. Still more preferably, the purified viral preparation comprises less than about 20 pg, and even more preferably, less than about 10 pg. Methods of measuring host cell nucleic acid levels in a viral sample are known in the art. Standardized methods approved or recommended by regulatory authorities such as the WHO or the FDA are preferred.

The invention also includes compositions comprising attenuated TTV. As used herein, attenuation refers to the decreased virulence of TTV in a porcine subject. Methods of attenuating viruses are known in the art. Such methods include serial passage of the virus in cultured cells as described above, until the virus demonstrates attenuated function. The temperature at which the virus is grown can be any temperature at which tissue culture passage attenuation occurs. Attenuated function of the virus after one or more passages in cell culture can be measured by one skilled in the art. Evidence of attenuated function may be indicated by decreased levels of viral replication or by decreased virulence in an animal model, as described above.

One particular method of producing an attenuated TTV includes passage of the virus in cell culture at suboptimal or “cold” temperatures and/or introduction of attenuating mutations into the TTV genome by random mutagenesis (e.g., chemical mutagenesis using for example 5-fluorouracil) or site specific-directed mutagenesis. Cold adaptation generally includes passage at temperatures between about 20° C. to about 32° C., and preferably between temperatures of about 22° C. to about 30° C., and most preferably between temperatures of about 24° C. and 28° C. The cold adaptation or attenuation may be performed by passage at increasingly reduced temperatures to introduce additional growth restriction mutations. The number of passages required to obtain safe, immunizing attenuated virus is dependent at least in part on the conditions employed. Periodic testing of the TTV culture for virulence and immunizing ability in animals can be used to readily determine the parameters for a particular combination of tissue culture and temperature.

TTV can also be attenuated by mutating one or more of the various viral regions, such as ORF1, ORF2 and/or ORF3, to reduce expression of the viral structural or nonstructural proteins. The attenuated TTV may comprise one or more additions, deletions or insertion in one or more of the regions of the viral genome.

Once attenuated, the virus is purified using techniques known in the art, such as described above with reference to inactivated viruses.

Subunit compositions can also be produced. For example, one or more immunogens derived from any of the viral genomic regions, such as derived from any of ORF1, ORF2 and/or ORF3, can be generated using recombinant methods, well known in the art. In this regard, oligonucleotide probes can be devised based on the sequences of the TTV genome and used to probe genomic or cDNA libraries for TTV genes encoding for the immunogens useful in the present invention. The genes can then be further isolated using standard techniques and, if desired, restriction enzymes employed to mutate the gene at desired portions of the full-length sequence. Alternatively, DNA sequences encoding the proteins of interest can be prepared synthetically rather than cloned. The DNA sequences can be designed with the appropriate codons for the particular amino acid sequence. In general, one will select preferred codons for the intended host if the sequence will be used for expression. The complete sequence is assembled from overlapping oligonucleotides prepared by standard methods and assembled into a complete coding sequence. See, e.g., Edge (1981) Nature 292:756; Nambair et al. (1984) Science 223:1299; Jay et al. (1984) J. Biol. Chem. 259:6311. TTV genes can also be isolated directly from viruses using known techniques, such as phenol extraction, and the sequence can be further manipulated to produce any desired alterations. See, e.g., Sambrook et al., supra, for a description of techniques used to obtain and isolate DNA.

Once coding sequences for the desired proteins have been prepared or isolated, they can be cloned into any suitable vector or replicon. Numerous cloning vectors are known to those of skill in the art, and the selection of an appropriate cloning vector is a matter of choice. Examples of recombinant DNA vectors for cloning and host cells which they can transform include the bacteriophage λ (E. coli), pBR322 (E. coli), pACYC177 (E. coli), pKT230 (gram-negative bacteria), pGV1106 (gram-negative bacteria), pLAFR1 (gram-negative bacteria), pME290 (non-E. coli gram-negative bacteria), pHV14 (E. coli and Bacillus subtilis), pBD9 (Bacillus), pIJ61 (Streptomyces), pUC6 (Streptomyces), YIp5 (Saccharomyces), YCp19 (Saccharomyces) and bovine papilloma virus (mammalian cells). See, generally, DNA Cloning: Vols. I & II, supra; Sambrook et al., supra; B. Perbal, supra.

The gene can be placed under the control of a promoter, ribosome binding site (for bacterial expression) and, optionally, an operator (collectively referred to herein as “control” elements), so that the DNA sequence encoding the desired protein is transcribed into RNA in the host cell transformed by a vector containing this expression construction. The coding sequence may or may not contain a signal peptide or leader sequence. If signal sequences are included, they can either be the native, homologous sequences, or heterologous sequences. Leader sequences can be removed by the host in post-translational processing. See, e.g., U.S. Pat. Nos. 4,431,739; 4,425,437; 4,338,397.

Other regulatory sequences may also be desirable which allow for regulation of expression of the protein sequences relative to the growth of the host cell. Regulatory sequences are known to those of skill in the art, and examples include those which cause the expression of a gene to be turned on or off in response to a chemical or physical stimulus, including the presence of a regulatory compound. Other types of regulatory elements may also be present in the vector, for example, enhancer sequences.

The control sequences and other regulatory sequences may be ligated to the coding sequence prior to insertion into a vector, such as the cloning vectors described above. Alternatively, the coding sequence can be cloned directly into an expression vector which already contains the control sequences and an appropriate restriction site.

In some cases it may be necessary to modify the coding sequence so that it may be attached to the control sequences with the appropriate orientation; i.e., to maintain the proper reading frame. It may also be desirable to produce mutants or analogs of the sequence of interest. Mutants or analogs may be prepared by the deletion of a portion of the sequence encoding the protein, by insertion of a sequence, and/or by substitution of one or more nucleotides within the sequence. Techniques for modifying nucleotide sequences, such as site-directed mutagenesis, are described in, e.g., Sambrook et al., supra; DNA Cloning, Vols. I and II, supra; Nucleic Acid Hybridization, supra.

It is often desirable that the polypeptides prepared using the above systems are fusion polypeptides. As with nonfusion proteins, these proteins may be expressed intracellularly or may be secreted from the cell into the growth medium.

Furthermore, plasmids can be constructed which include a chimeric gene sequence, encoding e.g., multiple TTV immunogens. The gene sequences can be present in a dicistronic gene configuration. Additional control elements can be situated between the various genes for efficient translation of RNA from the distal coding region. Alternatively, a chimeric transcription unit having a single open reading frame encoding the multiple antigens can also be constructed. Either a fusion can be made to allow for the synthesis of a chimeric protein or alternatively, protein processing signals can be engineered to provide cleavage by a protease such as a signal peptidase, thus allowing liberation of the two or more proteins derived from translation of the template RNA. The processing protease may also be expressed in this system either independently or as part of a chimera with the antigen and/or cytokine coding region(s). The protease itself can be both a processing enzyme and a vaccine antigen.

The expression vector is then used to transform an appropriate host cell. The molecules can be expressed in a wide variety of systems, including insect, mammalian, bacterial, viral and yeast expression systems, all well known in the art. For example, insect cell expression systems, such as baculovirus systems, are known to those of skill in the art and described in, e.g., Summers and Smith, Texas Agricultural Experiment Station Bulletin No. 1555 (1987). Materials and methods for baculovirus/insect cell expression systems are commercially available in kit form from, inter alia, Invitrogen, San Diego CA (“MaxBac” kit). Similarly, bacterial and mammalian cell expression systems are well known in the art and described in, e.g., Sambrook et al., supra. Yeast expression systems are also known in the art and described in, e.g., Yeast Genetic Engineering (Barr et al., eds., 1989) Butterworths, London.

A number of appropriate host cells for use with the above systems are also known. For example, mammalian cell lines are known in the art and include immortalized cell lines available from the American Type Culture Collection (ATCC), such as, but not limited to, Chinese hamster ovary (CHO) cells, HeLa cells, baby hamster kidney (BHK) cells, monkey kidney cells (COS), human embryonic kidney cells (e.g., HEK293), human hepatocellular carcinoma cells (e.g., Hep G2), Madin-Darby bovine kidney (“MDBK”) cells, as well as others. Similarly, bacterial hosts such as E. coli, Bacillus subtilis, and Streptococcus spp., will find use with the present expression constructs. Yeast hosts useful in the present invention include inter alia, Saccharomyces cerevisiae, Candida albicans, Candida mallow, Hansenula polymorpha, Kluyveromyces fragilis, Kluyveromyces lactis, Pichia guillerimondii,

Pichia pastoris, Schizosaccharomyces pombe and Yarrowia lipolytica. Insect cells for use with baculovirus expression vectors include, inter alia, Aedes aegypti, Autographa californica, Bombyx mori, Drosophila melanogaster, Spodoptera frugiperda, and Trichoplusia ni.

Depending on the expression system and host selected, the immunogens of the present invention are produced by growing host cells transformed by an expression vector under conditions whereby the immunogen of interest is expressed. The immunogen is then isolated from the host cells and purified. If the expression system provides for secretion of the immunogen, the immunogen can be purified directly from the media. If the immunogen is not secreted, it is isolated from cell lysates. The selection of the appropriate growth conditions and recovery methods are within the skill of the art.

The TTV immunogens may also be produced by chemical synthesis such as by solid phase or solution peptide synthesis, using methods known to those skilled in the art. Chemical synthesis of peptides may be preferable if the antigen in question is relatively small. See, e.g., J. M. Stewart and J. D. Young, Solid Phase Peptide Synthesis, 2nd Ed., Pierce Chemical Co., Rockford, Ill. (1984) and G. Barany and R. B. Merrifield, The Peptides: Analysis, Synthesis, Biology, editors E. Gross and J. Meienhofer, Vol. 2, Academic Press, New York, (1980), pp. 3-254, for solid phase peptide synthesis techniques; and M. Bodansky, Principles of Peptide Synthesis,

Springer-Verlag, Berlin (1984) and E. Gross and J. Meienhofer, Eds., The Peptides: Analysis, Synthesis, Biology, supra, Vol. 1, for classical solution synthesis.

The immunogens can be used to produce antibodies, both polyclonal and monoclonal. If polyclonal antibodies are desired, a selected mammal, (e.g., mouse, rabbit, goat, horse, etc.) is immunized with an immunogen of the present invention, or its fragment, or a mutated immunogen. Serum from the immunized animal is collected and treated according to known procedures. See, e.g., Jurgens et al. (1985) J. Chrom. 348:363-370. If serum containing polyclonal antibodies is used, the polyclonal antibodies can be purified by immunoaffinity chromatography, using known procedures.

Monoclonal antibodies to the immunogens, can also be readily produced by one skilled in the art. The general methodology for making monoclonal antibodies by using hybridoma technology is well known. Immortal antibody-producing cell lines can be created by cell fusion, and also by other techniques such as direct transformation of B lymphocytes with oncogenic DNA, or transfection with Epstein-Barr virus. See, e.g., M. Schreier et al., Hybridoma Techniques (1980); Hammerling et al., Monoclonal Antibodies and T-cell Hybridomas (1981); Kennett et al., Monoclonal Antibodies (1980); see also U.S. Pat. Nos. 4,341,761; 4,399,121; 4,427,783; 4,444,887; 4,452,570; 4,466,917; 4,472,500, 4,491,632; and 4,493,890. Panels of monoclonal antibodies produced against the TTV immunogen of interest, or fragment thereof, can be screened for various properties; i.e., for isotype, epitope, affinity, etc. Monoclonal antibodies are useful in purification, using immunoaffinity techniques, of the individual antigens which they are directed against. Both polyclonal and monoclonal antibodies can also be used for passive immunization or can be combined with subunit vaccine preparations to enhance the immune response.

TTV Formulations and Administration

The inactivated, attenuated or isolated TTV immunogens of the present invention can be formulated into compositions, such as vaccine or diagnostic compositions, either alone or in combination with other antigens, for use in immunizing subjects as described below. For example, the compositions can include additional immunogens from pathogens that cause disease in pigs, such as but not limited to, immunogens from porcine parvovirus, porcine circovirus, porcine reproductive and respiratory syndrome virus, swine influenza, pseudorabies virus, pestivirus which causes porcine swine fever, porcine lymphotropic herpesviruses (PLHV1 and PLHV2), Mycoplasma spp, Helicobacter spp, Campylobacter spp, Lawsonia spp,. Actinobacillus pleuropneumoniae, Haemophilus parasuis, Streptococcus spp, Pasteurella spp, Salmonella spp, E. coli, Clostridium spp, Eryspelothrix rhusiopathiae.

Methods of preparing such formulations are described in, e.g., Remington's Pharmaceutical Sciences, Mack Publishing Company, Easton, Pa., 18 Edition, 1990. Typically, the vaccines of the present invention are prepared as injectables, either as liquid solutions or suspensions. Solid forms suitable for solution in or suspension in liquid vehicles prior to injection may also be prepared. The preparation may also be emulsified or the active ingredient encapsulated in liposome vehicles. Vaccines suitable for oral delivery can also be readily formulated. The active immunogenic ingredient is generally mixed with a compatible pharmaceutical vehicle, such as, for example, water, saline, dextrose, glycerol, ethanol, or the like, and combinations thereof. In addition, if desired, the vehicle may contain minor amounts of auxiliary substances such as wetting or emulsifying agents and pH buffering agents.

Adjuvants which enhance the effectiveness of the vaccine may also be added to the formulation. Adjuvants may include for example, muramyl dipeptides, avridine, aluminum hydroxide, alum, Freund's adjuvant, incomplete Freund's adjuvant (ICFA), dimethyldioctadecyl ammonium bromide (DDA), oils, oil-in-water emulsions, saponins, cytokines, and other substances known in the art. Such adjuvants are well known and commercially available from a number of sources, e.g., Difco, Pfizer Animal Health, Newport Laboratories, etc.

TTV immunogens may also be linked to a carrier in order to increase the immunogenicity thereof. Suitable carriers include large, slowly metabolized macro-molecules such as proteins, including serum albumins, keyhole limpet hemocyanin, immunoglobulin molecules, thyroglobulin, ovalbumin, and other proteins well known to those skilled in the art; polysaccharides, such as sepharose, agarose, cellulose, cellulose beads and the like; polymeric amino acids such as polyglutamic acid, polylysine, and the like; amino acid copolymers; and inactive virus particles. TTV immunogens may be used in their native form or their functional group content may be modified by, for example, succinylation of lysine residues or reaction with Cys-thiolactone. A sulfhydryl group may also be incorporated into the carrier (or antigen) by, for example, reaction of amino functions with 2-iminothiolane or the N-hydroxysuccinimide ester of 3-(4-dithiopyridyl propionate. Suitable carriers may also be modified to incorporate spacer arms (such as hexamethylene diamine or other bifunctional molecules of similar size) for attachment of peptides.

Furthermore, the TTV immunogens may be formulated into vaccine compositions in either neutral or salt forms. Pharmaceutically acceptable salts include the acid addition salts (formed with the free amino groups of the active polypeptides) and which are formed with inorganic acids such as, for example, hydrochloric or phosphoric acids, or such organic acids as acetic, oxalic, tartaric, mandelic, and the like. Salts formed from free carboxyl groups may also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

Vaccine formulations will contain a “therapeutically effective amount” of the active ingredient, that is, an amount capable of eliciting an immune response in a subject to which the composition is administered. In the treatment and prevention of PRRSV infection, a “therapeutically effective amount” is readily determined by one skilled in the art using standard tests. The TTV immunogens will typically range from about 1% to about 95% (w/w) of the composition, or even higher or lower if appropriate. With the present vaccine formulations, 1 to 500 mg of active ingredient per ml, preferably 1 to 100 mg/ml, more preferably 10 to 50 mg/ml, such as 20 . . . 25 . . . 30 . . . 35 . . . 40, etc., or any number within these stated ranges, of injected solution should be adequate to raise an immunological response when a dose of 0.25 to 3 ml per animal is administered.

If an inactivated or attenuated preparation is used, the compositions will generally include 10² to 10¹² pfu, more particularly from 10⁴ to 10⁸ pfu, and preferably from 10⁵ to 10⁷ pfu of TTV.

To immunize a subject, the vaccine is generally administered parenterally, usually by intramuscular injection. Other modes of administration, however, such as subcutaneous, intraperitoneal and intravenous injection, are also acceptable. The quantity to be administered depends on the animal to be treated, the capacity of the animal's immune system to synthesize antibodies, and the degree of protection desired. Effective dosages can be readily established by one of ordinary skill in the art through routine trials establishing dose response curves. The subject is immunized by administration of the vaccine in at least one dose, and preferably two or more doses. Moreover, the animal may be administered as many doses as is required to maintain a state of immunity to infection.

Additional vaccine formulations which are suitable for other modes of administration include suppositories and, in some cases, aerosol, intranasal, oral formulations, and sustained release formulations. For suppositories, the vehicle composition will include traditional binders and carriers, such as, polyalkaline glycols, or triglycerides. Such suppositories may be formed from mixtures containing the active ingredient in the range of about 0.5% to about 10% (w/w), preferably about 1% to about 2%. Oral vehicles include such normally employed excipients as, for example, pharmaceutical grades of mannitol, lactose, starch, magnesium, stearate, sodium saccharin cellulose, magnesium carbonate, and the like. These oral vaccine compositions may be taken in the form of solutions, suspensions, tablets, pills, capsules, sustained release formulations, or powders, and contain from about 10% to about 95% of the active ingredient, preferably about 25% to about 70%.

Intranasal formulations will usually include vehicles that neither cause irritation to the nasal mucosa nor significantly disturb ciliary function. Diluents such as water, aqueous saline or other known substances can be employed with the subject invention. The nasal formulations may also contain preservatives such as, but not. limited to, chlorobutanol and benzalkonium chloride. A surfactant may be present to enhance absorption of the subject proteins by the nasal mucosa.

Controlled or sustained release formulations are made by incorporating the protein into carriers or vehicles such as liposomes, nonresorbable impermeable polymers such as ethylenevinyl acetate copolymers and Hytrel copolymers, swellable polymers such as hydrogels, or resorbable polymers such as collagen and certain polyacids or polyesters such as those used to make resorbable sutures. The TTV immunogens can also be delivered using implanted mini-pumps, well known in the art.

The TTV immunogens can also be administered via a carrier virus which expresses the same. Carrier viruses which will find use with the instant invention include but are not limited to the vaccinia and other pox viruses, adenovirus, and herpes virus. By way of example, vaccinia virus recombinants expressing the novel proteins can be constructed as follows. The DNA encoding the particular protein is first inserted into an appropriate vector so that it is adjacent to a vaccinia promoter and flanking vaccinia DNA sequences, such as the sequence encoding thymidine kinase (TK). This vector is then used to transfect cells which are simultaneously infected with vaccinia. Homologous recombination serves to insert the vaccinia promoter plus the gene encoding the instant protein into the viral genome. The resulting TK'recombinant can be selected by culturing the cells in the presence of 5-bromodeoxyuridine and picking viral plaques resistant thereto.

An alternative route of administration involves gene therapy or nucleic acid immunization. Thus, nucleotide sequences (and accompanying regulatory elements) encoding the subject TTV immunogens can be administered directly to a subject for in vivo translation thereof. Alternatively, gene transfer can be accomplished by transfecting the subject's cells or tissues ex vivo and reintroducing the transformed material into the host. DNA can be directly introduced into the host organism, i.e., by injection (see International Publication No. WO/90/11092; and Wolff et al. (1990) Science 247:1465-1468). Liposome-mediated gene transfer can also be accomplished using known methods. See, e.g., Hazinski et al. (1991) Am. J. Respir. Cell Mol. Biol. 4:206-209; Brigham et al. (1989) Am. J. Med. Sci. 298:278-281; Canonico et al. (1991) Clin. Res. 39:219A; and Nabel et al. (1990) Science 1990) 249:1285-1288. Targeting agents, such as antibodies directed against surface antigens expressed on specific cell types, can be covalently conjugated to the liposomal surface so that the nucleic acid can be delivered to specific tissues and cells susceptible to infection.

Diagnostics

Piglets suspected of having TTV and/or PRRSV infections may be tested for the same using standard procedures, well known in the art. As explained above, If pigs test positive for both TTV and PRRSV infection, they will have a greater propensity for developing PRRSVD, such as interstitial pneumonia.

Such diagnostic tests include standard electrophoretic and immunodiagnostic techniques, including immunoassays such as competition, direct reaction, or sandwich type assays. Such assays include, but are not limited to, Western blots; agglutination tests; enzyme-labeled and mediated immunoassays, such as ELISAs; biotin/avidin type assays; radioimmunoassays; immunoelectrophoresis; immunoprecipitation, immune fluorescence, IPMA, and virus isolation. See, e.g., Done et al., Br. vet. J. (1996):152-153; Oleksiewicz et al., Vet. Microbiol. 64:7-22. The reactions generally include revealing labels such as fluorescent, chemiluminescent, radioactive, enzymatic labels or dye molecules, or other methods for detecting the formation of a complex between the antigen and the antibody or antibodies reacted therewith.

The aforementioned assays generally involve separation of unbound antibody in a liquid phase from a solid phase support to which antigen-antibody complexes are bound. Solid supports which can be used in the practice of the invention include substrates such as nitrocellulose (e.g., in membrane or microtiter well form); polyvinylchloride (e.g., sheets or microtiter wells); polystyrene latex (e.g, beads or microtiter plates); polyvinylidine fluoride; diazotized paper; nylon membranes; activated beads, magnetically responsive beads, and the like.

Typically, a solid support is first reacted with a solid phase component (e.g., one or more antigens of interest, under suitable binding conditions such that the component is sufficiently immobilized to the support. Sometimes, immobilization of the antigen to the support can be enhanced by first coupling the antigen to a protein with better binding properties. Suitable coupling proteins include, but are not limited to, macromolecules such as serum albumins including bovine serum albumin (BSA), keyhole limpet hemocyanin, immunoglobulin molecules, thyroglobulin, ovalbumin, and other proteins well known to those skilled in the art. Other molecules that can be used to bind the antigens to the support include polysaccharides, polylactic acids, polyglycolic acids, polymeric amino acids, amino acid copolymers, and the like. Such molecules and methods of coupling these molecules to the antigens, are well known to those of ordinary skill in the art. See, e.g., Brinkley, M. A., Bioconjugate Chem. (1992) 3:2-13; Hashida et al., J. Appl. Biochem. (1984) 6:56-63; and Anjaneyulu and Staros, International J. of Peptide and Protein Res. (1987) 30:117-124.

After reacting the solid support with the solid phase component, any non-immobilized solid-phase components are removed from the support by washing, and the support-bound component is then contacted with a biological sample suspected of containing ligand moieties (e.g., antibodies toward the immobilized antigens) under suitable binding conditions. After washing to remove any non-bound ligand, a secondary binder moiety is added under suitable binding conditions, where the secondary binder is capable of associating selectively with the bound ligand. The presence of the secondary binder can then be detected using techniques well known in the art.

More particularly, an ELISA method can be used, where the wells of a microtiter plate are coated with the antigen(s). A biological sample containing or suspected of containing anti-TTV or PRRSV immunoglobulin molecules is then added to the coated wells. In assays where it is desired to use one microtiter plate, a selected number of wells can be coated with, e.g., a first antigen moiety, a different set of wells coated with a second antigen moiety, and so on. In the alternative, a series of ELISAs can be run in tandem. After a period of incubation sufficient to allow antibody binding to the immobilized antigens, the plate(s) can be washed to remove unbound moieties and a detectably labeled secondary binding molecule added. The secondary binding molecule is allowed to react with any captured sample antibodies, the plate washed and the presence of the secondary binding molecule detected using methods well known in the art.

Thus, in one particular embodiment, the presence of bound anti-TIN or anti-PRRSV antigen ligands from a biological sample can be readily detected using a secondary binder comprising an antibody directed against the antibody ligands. A number of useful immunoglobulin (Ig) molecules are known in the art and commercially available. Ig molecules for use herein will preferably be of the IgG or IgA type, however, IgM may also be appropriate in some instances. The Ig molecules can be readily conjugated to a detectable enzyme label, such as horseradish peroxidase, glucose oxidase, Beta-galactosidase, alkaline phosphatase and urease, among others, using methods known to those of skill in the art. An appropriate enzyme substrate is then used to generate a detectable signal. In other related embodiments, competitive-type ELISA techniques can be practiced using methods known to those skilled in the art.

Assays can also be conducted in solution, such that the viral proteins and antibodies specific for those viral proteins form complexes under precipitating conditions. In one particular embodiment, the antigen(s) can be attached to a solid phase particle (e.g., an agarose bead or the like) using coupling techniques known in the art, such as by direct chemical or indirect coupling. The antigen-coated particle is then contacted under suitable binding conditions with a biological sample suspected of containing antibodies for TTV and/or PRRSV. Cross-linking between bound antibodies causes the formation of particle-antigen-antibody complex aggregates which can be precipitated and separated from the sample using washing and/or centrifugation. The reaction mixture can be analyzed to determine the presence or absence of antibody-antigen complexes using any of a number of standard methods, such as those immunodiagnostic methods described above.

In yet a further embodiment, an immunoaffinity matrix can be provided, wherein a polyclonal population of antibodies from a biological sample suspected of containing anti-TTV and/or anti-PRRSV antibodies is immobilized to a substrate. In this regard, an initial affinity purification of the sample can be carried out using immobilized antigens. The resultant sample preparation will thus only contain anti-TTV and/or anti-PRRSV moieties, avoiding potential nonspecific binding properties in the affinity support. A number of methods of immobilizing immunoglobulins (either intact or in specific fragments) at high yield and having good retention of antigen binding activity, are known in the art. Not being limited by any particular method, immobilized protein A or protein G can be used to immobilize immunoglobulins.

Accordingly, once the immunoglobulin molecules have been immobilized to provide an immunoaffinity matrix, the antigens, having separate and distinct labels, are contacted with the bound antibodies under suitable binding conditions. After any non-specifically bound antigen has been washed from the immunoaffinity support, the presence of bound antigen can be determined by assaying for each specific label using methods known in the art.

Other assay methods include PCR, such as RT-PCR, a technique that amplifies RNAs by reverse transcribing the mRNA into cDNA, and then performing PCR. The PCR method for amplifying target nucleic acid sequences in a sample is well known in the art and has been described in, e.g., Innis et al. (eds.) PCR Protocols (Academic Press, NY 1990); Taylor (1991) Polymerase chain reaction: basic principles and automation, in PCR: A Practical Approach, McPherson et al. (eds.) IRL Press, Oxford; Saiki et al. (1986) Nature 324:163; as well as in U.S. Pat. Nos. 4,683,195, 4,683,202 and 4,889,818, all incorporated herein by reference in their entireties.

The fluorogenic 5′ nuclease assay, known as the TaqMan™ assay (see, e.g., Holland et al., Proc. Nail. Acad Sci. USA (1991) 88:7276-7280), is a powerful and versatile PCR-based detection system for nucleic acid targets. Hence, primers and probes derived from conserved regions of the TTV and PRRSV genomes can be used in TaqMan™ analyses to detect the presence of these viruses in a biological sample. For a detailed description of the TaqMan™ assay, reagents and conditions for use therein, see, e.g., Holland et al., Proc. Natl. Acad. Sci, U.S.A. (1991) 88:7276-7280; U.S. Pat. Nos. 5,538,848, 5,723,591, and 5,876,930, all incorporated herein by reference in their entireties.

The TTV and PRRSV sequences may also be used as a basis for transcription-mediated amplification (TMA) assays. TMA provides a method of identifying target nucleic acid sequences present in very small amounts in a biological sample. TMA is described in detail in, e.g., U.S. Pat. No. 5,399,491, the disclosure of which is incorporated herein by reference in its entirety.

The above-described assay reagents, including the immunogens, optionally immobilized on a solid support, can be provided in kits, with suitable instructions and other necessary reagents, in order to conduct immunoassays as described above. The kit can also contain, depending on the particular immunoassay used, suitable labels and other packaged reagents and materials (i.e. wash buffers and the like). Standard immunoassays, such as those described above, can be conducted using these kits.

3. EXPERIMENTAL

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### EXAMPLE 1

### Experimental Reproduction of PDNS in Porcine Circovirus Type 2 (PCV2)-negative Gnotobiotic Swine

In order to determine the role of TTV in PRRSV diseases, such as PDNS, the following experiment was conducted.

### Materials and Methods

### Animals and Experimental Design:

Gnotbiotic swine from all or part of four litters were used in this transmission study. Methods for derivation and husbandry of these piglets have been reported elsewhere (Krakowka S, Eaton K A (1996) in Advances in Swine in Biomedical Research II, eds Tumbleson M, Schook L (Plenum Press, NY), pp 779-810). All piglets were examined at least three times per day and clinically evident signs of disease were recorded for each piglet. Archived terminal sera from sows from that were used for derivation of gnotobiotic swine were also available for herd diagnostic evaluation.

### Transmission Experiment 1:

Collected plasma samples from 20 conventional swine, 14-16 weeks of age, (11 barrows, 9 gilts), were screened for PCV2 DNA viremia by PCR (McIntosh et al., Can. J. Vet. Res. (2006) 70:58-61); 18 were PCV2-negative; 2 were PCV2-positive. The latter two were discarded and a pool of the 18 remaining PCV2-negative plasma samples was made, designated as pass 0 (p0) and 8.0 ml of pooled plasmas was inoculated intraperitoneally (IP) into each of three 3 day-old gnotobiotic swine.

### Transmission Experiment 2:

For the second transmission experiment, a 10% (w/v) liver homogenate in Hank's minimal essential medium MEM) was made from one piglet from Experiment 1 that was terminated on post infection day (PID) 28. The homogenate was clarified by centrifugation and the supernatant was aliquoted into 2.0 ml amounts, designated as passage 1 (p1) and used as an intraperitoneal (IP) inoculum for piglets from two subsequent litters of gnotobiotic pigs (n=16). Of the latter, one was terminated on PID 5 when moribund, three were terminated on PID 7 (one was moribund), three on PIDs 13-14 (one was moribund), two on PID 21 and four on PID 32. Three separately housed gnotobiotic piglets were not inoculated and were used as uninfected control animals.

### Transmission Experiment 3:

For the third experiment, a gnotobiotic litter of 11 piglets was divided into separately-housed challenge groups. Group A piglets (n=3) received the PRRSV alone that had been recovered from pl homogenate by culture and one in vitro pass (PRRSVp1) on MARC cells. This PRRSV is a noncytopathogenic North American (NA) strain as determined by a combination of immuno-reactivity with NA-strain-specific monoclonal antibody and PRRSV-specific reverse transcriptase (rt) polymerase chain reaction (PCR) as described below. Group B piglets (n=4) received chloroform-extracted (C-E) (Feinstone et al., Infect. Immun. (1983) 41:816-821) fourth in vivo passage of TTV (TTVp4) and the PRRSV recovered from p2 inoculum. Group C piglets (n=4) received p1 homogenate used as inoculum for Transmission Experiment 2. One Group B piglet was terminated on PID 8 with clinical signs of wasting and respiratory distress; the ten remaining pigs while exhibiting transient anorexia and diarrhea, survived viral challenge and were terminated in PID 27 (Group A) or PID 28 (Groups B and C).

### Serology, Virology and Immunohistochemistry (IHC):

To construct an infectious disease history in the herd, 27 terminal sow sera collected from Caesarian derivations for gnotobiotic swine over the last 3 years were tested for antibodies to common swine pathogens by virus neutralization (encephalomyocarditis virus, EMC and transmissible gastroenteritis virus, TGE), agar gel immunoprecipitation, (swine influenza virus, SIV), hemagglutination inhibition assay (porcine parvovirus, PPV), ELISA (porcine reproductive and respiratory syndrome virus (PRRSV) and reverse transcriptase (rt) PCR for PRRSV viral RNA (Christopher-Hennings et al., J. Clin. Microbiol. (1995) 33:1730-1734) at The Animal Disease Diagnostic Laboratory, Ohio Department of Agriculture, Reynoldsburg, Ohio. Terminal sera from all experimentally infected gnotobiotes were tested for antibodies to these same viral pathogens. As well, terminal sera were screened for PCV2 by PCR assay (Lainson et al., J. Clin. Microbiol. (2002) 40:588-593) and for porcine torque teno virus genogroup 1 TTV DNAs by nested PCR (nPCR) using published primer sequences (Helie et al., Can. Vet. J. (1995) 36:648-660).

### Pathology:

Piglets were terminated on the PID intervals identified above. Gross lesions were photographed and tissue samples of peripheral lymph nodes, spleen, thymus, bone marrow, lung liver, kidney and ileum were collected into tissue cassettes, fixed for 24 hrs in 100% cold ethanol and then processed by routine histologic methods for embedding in paraffin and sectioning. Five-micron thick section replicates were stained with hematoxylin and eosin (HE), Jones' silver and PAS stains and by immunohistochemistry (IHC) methods for PCV2 nucleocapsid protein and porcine fibrinogen/fibrin by published methods (Krakowka et al., Vet. Pathol. (2000) 37:274-282; Krakowka et al., Vet. Pathol. (2001) 38:31-42; Krakowka et al., Virol. Immunol. (2002) 15:567-582).

### Results

### Herd Disease History:

An infectious disease history profile (2003 through the spring of 2006) of the source herd was reconstructed by assessment of antibody titers to common swine pathogens. All 27 sow sera tested were antibody-positive for PCV2 and PPV, but negative for PCV2 viremia by PCR. By serology, the herd was negative for TGE and variably positive for EMC viruses. A few sow samples were PRRSV-positive by ELISA assay but these were low titers, in sporadic incidence (0-1 sow per year) and attributable to residual vaccination-associated titers in these animals. The herd sero-converted to SIV in mid 2005, although clinically evident respiratory disease in swine was never expressed. All but one sow serum was swine genogroup 1 TTV DNA-positive by nPCR.

### Clinical and Pathologic Findings, Transmission Experiments 1-3

### Clinical Findings:

All three piglets in Transmission Experiment 1 remained clinically normal throughout the course of the four-week experiment. In contrast, all inoculated piglets of Transmission Experiment 2 that received pl homogenate derived from Transmission Experiment 1 developed clinical signs of disease. One day after IP infection with p1, all 16 piglets became mildly anorexic, sluggish and lethargic when handled. These signs persisted for the next 4-5 days. Mild dyspnea was noted on PID 3 and continued through PIDs 7-10. Diarrhea (loose stool) developed after PID 3 and persisted for 7-10 days thereafter although the severity varied between piglets in that some still evidenced diarrhea at termination on PID 32; others did not. Of the 16 inoculated piglets in Experiment 2, three (PIDs 5, 7 and 13) became recumbent and unresponsive and were terminated. One piglet of Group B in Transmission Experiment 3 developed severe diarrhea and dyspnea on PID 6; it was terminated on PID 8. The remaining piglets in this litter, except for transient diarrhea in piglets of Group B and C, survived the viral challenge and were terminated on PIDs 27 and 28. Uninfected control piglets remained clinically normal throughout the experiment.

### Pathology Findings, Transmission Experiment 1:

The gross findings in the two piglets terminated on PID 28 were mild generalized lymphadenopathy associated particularly by prominent lymphoid follicle development, moderate thymic atrophy and pale-to-tan livers. Histologically, lymphofollicular hyperplasia and thymic atrophy were confirmed and modest lymphocytic-histiocytic inflammatory cell infiltrates into hepatic sinusoids (mild multifocal nonsuppurative hepatitis) were seen. Minimal lymphadenopathy and lymphocytic hepatitis were seen in the third piglet.

### Pathology Findings, Transmission Experiment 2:

For convenience, the gross and histologic findings in the second transmission experiment were subdivided by the post infection day (PID) of termination, grouped together and presented by PID interval.

### PIDs 5-7:

A constellation of gross and histologic lesions was seen in these piglets, summarized as follows. A systemic circulatory disturbance was evident and manifest as mild subcutaneous edema, mild icterus, and thin poorly clotted blood. A mild generalized lymphadenopathy and thymic atrophy characterized the changes in peripheral lymph nodes. The lungs were tan in color and did not collapse when the thorax was opened. The livers were mottled and yellow to tan and the kidney cortex of one piglet had multifocal petechial hemorrhages. Histologically, lymphoid tissues were reactive, specifically follicular hyperplasia and the thymi exhibited T cell depletion (thymic atrophy). Hepatocytes exhibited cellular swelling and degeneration and subtle but diffuse and “active” non-suppurative inflammatory infiltrates were detected throughout the sections, most prominent in the hepatic sinusoids. The alveolar walls and capillary vasculature of the lungs contained mononuclear inflammatory cells, scattered neutrophils and proteinic deposits consistent with a morphologic diagnosis of acute diffuse (mild) interstitial pneumonitis. Subtle but distinct disruptions of the endothelial lining of larger pulmonary vessels were occasionally identified and were associated with poorly formed intravascular micro-thrombi. Hemorrhages were confirmed in the kidneys and PAS-, Jones silver stain- and PTAH-positive plasma protein (fibrinoid) deposits distended renal glomeruli. These deposits stained positively for fibrinogen/fibrin by IHC.

### PIDs 13-14:

A similar but more severe constellation of gross and histologic lesions as were seen in piglets above were identified in these piglets except that they were more severe. In addition, bilaterally symmetrical subcutaneous hemorrhages were seen in the ventral abdomen and hams of one PID 13 piglet; the latter was associated with tail ulceration. Bilaterally symmetrical hemorrhages were seen in the inguinal region of a second piglet; anasarca, icterus, anemia and hemostatic defects were also observed. Livers were mottled and the lungs were distended and did not collapse when the thorax was opened. Renal cortical hemorrhages and accentuation of renal glomeruli (white cortical foci) were present in two piglets while accentuation of glomeruli without hemorrhages was seen in the third piglet.

Histologically, the subcutaneous skin lesions consisted of a lymphocytic and histiocytic inflammatory vasculitis and per-vasculitis, sub-epidermal edema and micro-hemorrhages. Livers contained foci of inflammatory cells that were not organized and similar to the active hepatitis lesion seen on PID 7. The lungs were dramatically altered from the normal. Alveolar walls were markedly distended with a mixed inflammatory cell infiltrate consisting of lymphocytes, plasma cells, macrophages and occasional neutrophils (PMNs) and closely resembled published photomicrographs of PRRSV pneumonia in gnotobiotic pigs (Rossow et al., Vet. Pathol. (1995) 32:361-373). As well, alveolar spaces frequently contained brightly stained refractive eosinophilic proteinic hyaline-like deposits containing an admixture of mononuclear inflammatory cells and other cellular debris for fibrin/fibrinogen by IHC. In the kidneys, the cortical hemorrhages were confirmed and renal glomeruli exhibited prominent membranous thickening, again attributable to accumulations of proteins including fibrinogen/fibrin deposits into or on glomerular basement membranes. As well, foci of interstitial inflammation (lymphocytes, plasma cells and macrophages) were also present. Sections of lymph nodes demonstrated lymphoid hyperplasic (activation), chiefly of B cell germinal centers.

### PID 21:

Two piglets terminated on PID 21 were clinically normal except for residual diarrhea. Gross and histologic lesions were again present in the kidneys and lungs but were of decreased severity when compared to the changes seen on PIDs 13/14. Grossly, there was no evidence of subcutaneous hemorrhages, yet mild subcutaneous edema was present in one; both piglets exhibited thin watery and poorly clotted blood. Peripheral lymph nodes were enlarged, thymi were atrophic, livers were normal but the lungs were firm and did not collapse when the thorax was opened. In general, histologic changes in tissue reflected gradual resolution of acute lesions in the kidney, lung and liver present on PIDs 7 through 14. In the liver, numerous foci of infiltrating lymphocytes, plasma cells and histiocytes were seen, the latter beginning to organize into granulomatous foci around regional areas of regional hepatocyte loss and sinusoidal expansion. In the kidneys, foci of interstitial fibrosis and accumulations of mononuclear inflammatory cells were seen in association with segmental tubular dilation. Renal glomeruli still contained proteinic material and stained strongly for fibrinogen/fibrin by IHC but were in the developing segmental scarring and fibrosis. The lungs were affected with resolving interstitial pneumonia. Aside from the severe thymic atrophy, both piglets demonstrated mild activation of lymphoid tissues associated with germinal center development and proliferation of histiocytes in the lymphoid sinusoidal areas.

### PID 32:

By PID 32, gross lesions in these piglets were minimal and consisted of moderate generalized lymphadenopathy (4 of 4), mild thymic atrophy (2 of 4) and pale or tan liver (3 of 4). Kidneys were grossly normal. Histologically, mild interstitial pneumonia was seen in 3 of 4 piglets. In the liver, modest multifocal accumulations of lymphocytes, monocytes and plasma cells were seen. Renal lesions in all four piglets were multi-focally distributed and varied in intensity amongst the four. Segmental to complete glomerular sclerosis was evident in all 4 pigs; the IHC stain for fibrinogen/fibrin deposits was equivocal. The number of affected glomeruli varied from a high of roughly 10% to a low of 1%. Accompanying this lesion were multifocally distributed adjacent areas of interstitial fibrosis and segmental tubular dilation and lymphoplasmacytic cellular infiltrations.

### Transmission Experiment 3:

All three gnotobiotic piglets inoculated with PRRSV alone (Group A) were clinically normal at termination on PID 27. Mild thymic atrophy and generalized lymphadenopathy was grossly evident and was subsequently confirmed by histological evaluation. The livers were of normal size and coloration and the lungs were grossly normal, pink and well aerated. No evidence for renal or cutaneous hemorrhages was discerned. In contrast, all piglets in infection Groups B and C developed clinical signs of disease as seen in Experiment 2 above. One Group B piglet developed respiratory distress on PID 6, became anorexic and was terminated on PID 8. In that piglet, mild lymphadenopathy, thymic atrophy and bilateral renal cortical hemorrhages were seen. The 3 remaining piglets of Group B were terminated on PID 28 and, as a group, expressed the same constellation of gross and histological lesions seen in Group C piglets of Transmission Experiment 3 and the PID 32 piglets above. All had mottled and/or mottled and pale livers, two piglets of Group B had mild lobular interstitial pneumonia and prominent glomeruli associated with corticomedullary hemorrhages. Generalized lymphadenopathy and nodular lymphoid hyperplasia were present in all piglets of Groups B and C. Histologic findings in these three piglets were similar to Experiment 2 above. That is, lymphoid activation characterized by systemic B cell (germinal center) formation was prominent as was zonal T cell proliferation and reticuloendothelial proliferation. The lungs were moderately to severely affected with interstitial pneumonia, similar to the PRRSV alone pigs of Group A. The renal glomeruli in the kidneys of all Group B and C were moderately to severely affected with membranous glomerulonephropathy; the three surviving piglets of Group B had multifocally distributed moderate to severe renal glomerular sclerosis (fibrosis).

### Serology and Virology:

Plasma samples collected from all piglets after PID 7 contained TTV DNAs by nPCR. None of the terminal serum samples from the pigs contained PCV2 viral DNAs when examined by PCR (Krakowka et al., Virol. Immunol. (2002) 15:567-582). As well, tissue section replicates from all infected piglets were tested for the presence of PCV2 viral nucleocapsid by IHC; all were negative. Other serologic and virologic results are summarized below. All piglets inoculated with p1 sero-converted to PRRSV antigen(s) by PID 21. Terminal sera tested for PRRSV RNAs by rtPCR were all positive. Two of three piglets inoculated with the pooled plasma samples (p0) contained PRRSV antibodies and TTV DNAs. Terminal sera from all piglets except a piglet terminated on PID 8 were PRRSV antibody-positive; the PID 8 piglet of Transmission Experiment 3 was also PRRSV RNA-positive by rtPCR. Terminal sera were tested for other viral pathogens of swine including TGE, PPV and EMC (data not shown); all were negative.

To the best of the inventors' knowledge, this is the first report describing the experimental reproduction of both the cutaneous and the renal lesions characteristic of PDNS in swine. There is one report that describes necrotizing vascular lesions in the kidneys of SPF swine experimentally infected with PRRSV (Cooper et al., Vet. Diag. Invest. (1997) 9:198-201). In these series, all PDNS-affected gnotobiotes were PCV2-negative as assessed by combinations of negative serology, negative PCR and lack of PCV2 nucleocapsid protein in any of the tissues. Rather, these experiments strongly associate PDNS with PRRSV and TTV co-infections. That TTV appears to be critical for expression of PDNS lesions is indicated by its presence (by nPCR) in p0 and p1 inocula and its intentional inclusion with PRRSV alone in Transmission Experiment 3. The source of PRRSV was likely one or more of the plasma samples (p0) collected from clinically healthy feeder pigs. After initiation of these experiments, further investigations into the cause of the illness in the source herd confirmed that shortly after plasma collections, the herd became PRRSV-positive, even though an aggressive vaccination program for PRRSV was in place at this facility.

The gross and histologic lesions identified as diagnostic for PDNS have been attributed to combinations of primary segmental vasculitis (Thibault et al., Vet. Pathol. (1998) 35:108-116; Thomson et al., J. Vet. Med. A Physiol. Pathol. Clin. Med. (2002) 49:430-437) and associated micro-thrombosis and/or immune complex phenomena (Gresham et al., Vet. Rec. (2000) 146:40-43; Sierra et al., Eur. J. Vet. Pathol. (1997) 3:63-70; Smith et al., Vet. Rec. (1993) 132:47; Thomson et al., J Vet. Med. A Physiol: Pathol. Clin. Med. (2002) 49:430-437). In the Canadian field experience (Helie et al., Can. Vet. J. (1995) 36:648-660; Thibault et al., Vet. Pathol. (1998) 35:108-116), the vasculitis detected in PRRSV-infected swine was widespread and associated with vascular lesions in most organ systems; the TIN status of these pigs is not known. In our experimental series, vascular disease sufficiently severe as to result in vasculitis and hemorrhages restricted to the skin and renal cortices. It is difficult to ascribe the glomerular lesions produced in affected gnotobiotes as due to an immune complex inflammatory insult per se as these lesions were seen as early as PID 7 and were of maximal intensity by PID 13/14, time intervals well before a significant immune response would be expected. In addition, piglets did not sero-convert to PRRSV antigens until PID 21, suggesting that if immune complexes were associated with circulating PRRSV antigen(s) and antibodies, glomerular deposition of proteins must have occurred before antibodies to PRRSV could have been produced in sufficient quantity to participate in the genesis of glomerular lesions. Finally, there was a paucity of infiltration of glomeruli by neutrophils that are classically associated with acute immune complex phenomena. This said, the lesions of PDNS in gnotobiotic swine were mild when compared to described field cases of severe PDNS. While the reason(s) for this are unknown, it is likely that the microbe-free status of our animals combined with the general lack of immunologic activation associated with gnotobiotic conditions could account for these severity differences.

While PRRSV is ordinarily thought to replicate in cells of monocyte and histiocyte lineages including macrophages, a number of authors have found PRRSV antigen(s) and RNAs in other cell types such as cardiomyocytes and vascular endothelia. PRRSV is notorious for persistent and intermittent viremia and appears able to co-exist in subpopulations of infected swine in spite of protective levels of PRRSV antibodies (Rossow et al., Vet. Pathol. (1995) 32:361-373). Similarly, PCV2 proteins and DNAs are prominent in cells of monocyte lineage (macrophages, histiocytes, dendritic cells, Kupffer cells) but may also present as a pantropic infection in epithelial cells (hepatocytes, renal tubules, respiratory epithelium) and endothelia (Allan and Ellis, J. Vet. Diag. Invest. (2000) 12:3-14; Krakowka et al., Vet. Pathol. (2000) 37:274-282; Krakowka et al., Vet. Pathol. (2001) 38:31-42; Krakowka et al., Virol. Immunol. (2002) 15:567-582); Segales and Domingo, Vet. Quart. (2002) 24:109-124). While vascular disease may be attributable to PRRSV, such manifestations are uncommon in PCV2-infected swine.

The search for the presumed infectious cause of PDNS has led investigators to consider all of the major swine viral pathogens and certain swine bacterial pathogens as candidate agents for PDNS. Some investigators have reported that PDNS glomeruli contain IgG and complement, findings strongly supportive of an immune complex etiology (Gresham et al., Vet. Rec. (2000) 146:40-43; Segales et al. (2002) Porcine dermatitis and nephropathy syndrome. in Trends in Emerging Viral Infections of Swine, eds Morilla A, Yoon K-J, Zimmerman J J (Iowa State University Press, Ames, Iowa), pp 313-318; Sierra et al., Eur. J. Vet. Pathol. (1997) 3:63-70; Smith et al., Vet. Rec. (1993) 132:47; Thomson et al., J. Vet. Med. A Physiol. Pathol. Clin. Med. (2002) 49:430-437). In the Canadian field experience (Helie et al., Can. Vet. J. (1995) 36:648-660; Thibault et al., Vet. Pathol. (1998) 35:108-116). The specificity(ies) of the IgG has not been elucidated but they are clearly not associated with PCV2 antigen-antibody complex deposition in these sites.

Others have suggested that the bulk of the protein that accumulates in glomeruli originate from acute phase plasma proteins and protein components of the coagulation cascade (Done et al., In Pract. (2001) 14-25; Drolet et al., Swine Health Prod. (1999) 8:283-285; Segales et al. (2002) Porcine dermatitis and nephropathy syndrome. in Trends in Emerging Viral Infections of Swine, eds Morilla A, Yoon K-J, Zimmerman J J (Iowa State University Press, Ames, Iowa), pp 313-318). In either case, subsequent acute inflammatory responses further damage the structural components of glomeruli such that scarring and fibrosis are expected to develop during convalescence. The case for PRRSV involvement is strong as others have described vascular disease associated with PRRSV (Cooper et al., Vet. Diag. Invest. (1997) 9:198-201; Helie et al., Can. Vet. J. (1995) 36:648-660; Thibault et al., Vet. Pathol. (1998) 35:108-116) and PRRSV is known to infect vascular endothelia. Yet, in all published studies of experimental PRRSV infections, PDNS has never been described as a consequence of PRRSV challenge. Further, PDNS is known to occur in PRRSV-free herds (Segales et al. (2002) Porcine dermatitis and nephropathy syndrome. in Trends in Emerging Viral Infections of Swine, eds Morilla A, Yoon K-J, Zimmerman J J (Iowa State University Press, Ames, Iowa), pp 313-318) and also occurs in countries free of PRRSV infection (Segales et al. (2002) Porcine dermatitis and nephropathy syndrome. in Trends in Emerging Viral Infections of Swine, eds Morilla A, Yoon K-J, Zimmerman J J (Iowa State University Press, Ames, Iowa), pp 313-318; Smith et al., Vet. Rec. (1993) 132:47). All inoculated piglets of Transmission Experiments 2 and 3 were TTV viremia-negative prior to challenge with p1 or TTVp4 and became viremic for genogroup 1 TTV DNAs in serum as determined by nested PCR by PID 7-14.

Rather than directly implicating a single infectious agent as the cause of PDNS, our data suggest that the primary underlying pathogenic mechanism for development of PDNS is a rapid-onset systemic coagulation defect or disseminated intravascular coagulation (DIC). The provisional diagnosis of DIC is supported by the findings of small vessel hemorrhages in the dermis and kidney cortices, prolonged bleeding from jugular venipuncture sites, the failure of blood coagulation during necropsy and the severe thrombocytopenia (<10,000 platelets/d1, data not shown) demonstrable in several terminal on clotted blood samples. Likely, the exudation of fibrin into alveolar spaces as a component of PRRSV-associated pneumonia (Rossow et al., Vet. Pathol. (1995) 32:361-373) as did vascular endothelial damage and micro-thrombosis all contributed to the poor coagulability of the blood from these piglets. In fact, the name “nephropathy” rather than “nephritis” speaks directly to the confusion regarding the nature of PDNS-specific renal glomerular lesions. In our series, renal glomeruli stained strongly with the Jones silver stain for basement membranes and strongly with an anti-sera reactive with porcine fibrinogen/fibrin on PID 7 and variably with anti-swine IgG reagents from PIDs 13/14 onward indicating that deposition of plasma proteins precedes deposits of IgG. These findings, combined with obvious coagulation defects identified in piglets strongly suggest that DIC contributed directly to the morbidity in three piglets (PIDs 5, 13 and 14) and was doubtless responsible for the clinical signs of depression, anorexia and diarrhea seen in all pigs inoculated with p1.

Thus, methods for treating, preventing and diagnosing PRRSV-associated infection are described, as well as compositions for use with the methods. Although preferred embodiments of the subject invention have been described in some detail, it is understood that obvious variations can be made without departing from the spirit and the scope of the invention as described herein.

1. A method of treating or preventing a PRRSV-associated disease (PRRSVD) in a porcine subject comprising administering to said subject a therapeutically effective amount of a composition comprising a pharmaceutically acceptable vehicle and at least one porcine Torque teno virus (TTV) immunogen selected from an inactivated immunogenic porcine TTV, an attenuated immunogenic porcine TTV or one or more isolated immunogenic porcine TTV polypeptides.

2. The method of claim 1, wherein the composition comprises an inactivated immunogenic porcine TTV.

3. The method of claim 1, wherein the composition comprises an attenuated immunogenic porcine TTV.

4. The method of claim 1, wherein the composition comprises one or more isolated immunogenic porcine TTV polypeptides.

5. The method of claim 1, wherein the composition further comprises an adjuvant.

6. The method of claim 1, wherein the PRRSVD is a respiratory disease.

7. The method of claim 6, wherein the PRRSVD is interstitial pneumonia.

8. The method of claim 1, wherein the PRRSVD is a reproductive disease.

9. The method of claim 1, wherein the PRRSVD is porcine dermatitis and nephropathy syndrome (PDNS).

10. A method of determining the propensity of a porcine subject to acquire a PRRSVD comprising determining whether the subject is infected with both TTV and PRRSV.

11. The method of claim 10, wherein the PRRSVD is interstitial pneumonia.

12. The method of claim 10, wherein the PRRSVD is a reproductive disease.

13. The method of claim 10, wherein the PRRSVD is PDNS.

14. A method for evaluating the ability of a vaccine to prevent a PRRSVD comprising:
(a) administering to a porcine subject a candidate vaccine;
(b) exposing the porcine subject from step (a) to a porcine TTV isolate and a porcine PRRSV isolate in amounts sufficient to cause infection in an unvaccinated subject; and
(c) observing the incidence of PRRSVD in the porcine subject, thereby evaluating the ability of the candidate vaccine to prevent PRRSVD.

15. The method of claim 14, wherein the porcine subject is a young TTV-negative and PRRSV-negative piglet, a barrier-raised specific pathogen-free piglet, or caesarian-delivered piglet.

16. The method of claim 14, wherein the PRRSVD is a respiratory disease.

17. The method of claim 16, wherein the respiratory disease is interstitial pneumonia.

18. The method of claim 14, wherein the PRRSVD is a reproductive disease.

19. The method of claim 14, wherein the PRRSVD is PDNS.

20. A method of identifying a compound capable of treating a PRRSVD, said method comprising:
(a) exposing a young TTV-negative and PRRSV-negative piglet, a barrier-raised specific pathogen-free piglet, or caesarian-delivered piglet to a porcine TTV isolate and a PRRSV isolate in amounts sufficient to cause infection in said piglet;
(b) delivering a compound or series of compounds to said infected piglet; and
(c) examining the piglet from step (b) for the presence or loss of TTV and PRRSV, and/or the development, inhibition, or amelioration of PRRSVD symptoms relative to an untreated TTV-infected and PRRSV-infected piglet.
